(19) United States
(12) Patent Application Publication
(10) Pub. No.: US 2022/0378848 A1
(43) Pub. Date: Dec. 1, 2022

(54) COMPOSITIONS AND METHODS OF USING MULTIPOTENT STEM CELLS TO REDUCE DISEASE AND ENHANCE WELLNESS

(71) Applicants: Jill WADE, Frisco, TX (US); Xerxez CALILUNG, Irvine, CA (US); RELEVANCE TOTAL HEALTH, Frisco, TX (US)

(72) Inventors: Jill WADE, Frisco, TX (US); Xerxez CALILUNG, Irvine, CA (US)

(21) Appl. No.: 17/630,862

(22) PCT Filed: Jul. 29, 2020
(86) PCT No.: PCT/US2020/044050

§ 371 Date: Jan. 27, 2022

Related U.S. Application Data
(60) Provisional application No. 62/879,943, filed on Jul. 29, 2019.

(51) Int. Cl. A61K 35/545; A61K 35/16; A61P 1/00; A61C 17/00; A61C 19/04; A61K 9/00

(52) U.S. Cl. CPC A61K 35/545; A61K 35/16; A61P 1/00; A61C 17/005; A61C 19/043; A61K 9/0019

(57) ABSTRACT

Provided herein are methods of using multipotent stem cells in treatment or prophylaxis of disease, in a system for improving general health and overall wellness. The disclosure provides a method in which multipotent stem cells are intravenously administered within the oral cavity, which has a rapid effect on symptoms of inflammation and/or infection, not only treating periodontal diseases, but also reducing bacterial load, pain, bone loss, etc. as well as stimulating new bone growth and achieving other hallmarks of improvement in health and overall systemic wellness. In some instances, a sample of a bodily fluid is drawn from the patient before injection to measure baseline levels of several biomarkers to obtain the patient's status on the spectrum of health and wellness and/or to compare post-treatment effects of the injected stem cells, to measure improvement, and to develop a treatment plan for enhancing multisystem wellness.

### CROSS REFERENCE TO RELATED APPLICATIONS

This application claims benefit under 35 U.S.C. § 119(e) to application Ser. No. 62/879,943 filed 29 Jul. 2019, the contents of which are incorporated herein by reference in their entirety.

### TECHNICAL FIELD

The present disclosure is generally directed to the field of improving health and enhancing wellness. Specifically, multipotent stem cells are used to combat disease or prevent its progression, fight infection, reduce inflammation, prevent tissue degeneration and enhance a patient's overall wellness. Provided herein are compositions and methods for treating disease using stem cell based therapies.

### BACKGROUND

Periodontal disease is a common chronic condition affecting the majority of Americans, usually 30 years of age and older. Microorganisms that cause periodontal disease can enter the general circulation causing a bacteraemia, often resulting in adverse systemic effects and, in some cases, promoting endocarditis and/or cardiovascular disease. Periodontal disease was found to be independently associated with arterial disease, and it is now believed that periodontal disease, due to high-risk pathogens, is a contributory cause of atherosclerosis. Atherosclerosis remains the largest cause of death and disability. Studies indicate that the adverse cardiovascular effects from periodontal disease can be attributed to the presence of several kinds of high-risk bacteria in the blood: Aggregatibacter actinomycetemcomitans (Aa), Porphyromonas gingivalis (Pg), Tannerella forsythia (Tf), Treponema denticola (Td) and/or Fusobacterium nucleatum (Fn). (Bale, et al., 2016, Postgrad. Med. J. 0:1-6).

Three essential elements are now recognized components of the pathogenesis of atherosclerosis, and are referred to as the “atherogenic triad”. These are: (1) increased serum lipoprotein concentration, with each lipoprotein containing apolipoprotein B (ApoB); (2) dysfunction and permeability of the endothelium, allowing monocytes to adhere to the endothelium and to penetrate into the intima along with lipoproteins; and (3) binding of ApoB to proteoglycans derived from migratory smooth muscle cells (SMCs), as well as conversion of monocytes to macrophages, which become foam cells. Interestingly, the atherogenic triad intensifies in the presence of high-risk periodontal pathogens. In the presence of high-risk periodontal pathogens, each of the three parts of the atherogenic triad are adversely enhanced causing greater atherogenesis, such that (1*) the concentration of ApoB is increased; (2*) the endothelial dysfunction and permeability are enhanced by lipopolysaccharides (LPS); and (3*) the binding of lipoproteins is enhanced by increased migratory SMCs, which enrich the intima with proteoglycans (Bale, et al., 2016, Postgrad. Med. J. 0:1-6).

Periodontal infections and associated inflammation can trigger a variety of systemic diseases and conditions, including cardiovascular disease and stroke. Many patients with periodontitis demonstrate elevated levels of anticardiolipin antibodies (“aCL” or “anti-CL”). Anti-CL are most commonly found in patients with autoimmune diseases such as scleroderma, systemic lupus erythematosis (SLE) or antiphospholipid syndrome (APS; also known as Hughes syndrome). It has been observed that patients with autoimmune diseases, aCL and/or APS have increased risk of (and in many cases, experience accelerated) atherosclerosis, and have a higher than normal incidence of venous thromboembolism, arterial thrombosis, adverse pregnancy outcome (fetal involution) and obstetric morbidities, and are at increased risk of accelerated atherosclerosis, myocardial infarction (MI), angina, valvular heart disease and stroke. APS is an acquired autoimmune thrombotic disorder in which antiphospholipid antibodies which are thought to activate endothelial cells, monocytes and platelets resulting in increased synthesis of tissue factors and thromboxane A2 causing thrombosis in the vascular bed. Vascular endothelial cell dysfunction mediated by antiphospholipid antibodies (against both “self” phospholipids, as well as non-self/pathogenic phospholipids) and subsequent complement system activation play a role in APS pathogenesis. Improved understanding of their pathogenic function could help in the risk stratification of patients. (Corban, et al., 2017, J. Am. Coll. Cardiol. 69(18):2317-2330).

A study of patients clinically characterized as to their periodontal disease status were assessed their serum levels of beta2-glycoprotein I(β2GP1)-dependent anti-cardiolipin autoantibodies. The prevalence of patients with chronic periodontitis (CP) and generalized aggressive periodontitis (GAgP) positive for anti-CL (16.2% and 19.3%, respectively) was greater than that in healthy controls (NP) and localized aggressive periodontitis (LAgP) patients (6.8% and 3.2%). Patients with these autoantibodies demonstrated increased pocket depth (PD) and attachment loss (AL) compared with patients lacking the antibodies. These data indicated that patients with generalized periodontitis have elevated levels of autoantibodies reactive with phospholipids. These antibodies could be involved in elevated risk for stroke, atherosclerosis, or pre-term birth in periodontitis patients. Previous studies have indicated that a significant proportion of individuals with periodontitis demonstrate elevated serum concentrations of aCl (Schenkein et al., 2003, J. Dent. Res. 82(11):919-922).

Some studies have used either Streptococcus mutans titers or past caries history, such as dmft or dmfs (deciduous teeth), and DMFT or DMFS (permanent teeth), to select the high risk and/or caries-active subjects. A problem associated with these inclusion criteria is the subjectiveness associated with the actual diagnoses, as well as lack of agreement on what number constitutes a caries-prone individual. This uncertainty about identification of individuals who have a high potential for future caries development is exemplified in a divided study that was partitioned into “high-risk” (S. mutans titer-based), and “caries-active” (DMFS-based) subjects. The same treatment agent gave a 33% reduction in new caries in the “high-risk” group and a 9% increase in the “caries-active” group (U.S. Dept. of Health & Human Services. National Institutes of Health Consensus Development Conference Statement. Diagnosis and Management of Dental Caries Throughout Life (2001)). In addition to the confusion about what inclusion criteria best identify those with the highest projected rates of cariogenesis, the assessment report notes that neither approach to caries risk assessment has ever been validated.

Thus, to date, treatments for APS are insufficient and lacking, and symptoms are addressed in a piecemeal fashion. A need remains for effective therapies and a systematic approach to address and treat, ameliorate or prevent APS and other immune or inflammatory diseases or disorders. The present disclosure addresses that need by providing a system for enhancing global, multisystem health and wellness starting at the top of the gastrointestinal tract: the oral cavity of a dental patient.

### SUMMARY

Generally disclosed herein are compositions and methods of treating, ameliorating, reducing risk of, reducing symptoms of an immune and/or inflammatory disorder in a patient/subject, comprising intravenously administering into the oral vasculature of the patient/subject an effective dose of a composition comprising or consisting essentially of multipotent stem cells as a therapeutic agent to treat the immune and/or inflammatory disorder.

Provided is a systematic program employing a multiparameter matrix to assess a series of indicators of a patient's baseline health and wellness status, scoring/quantifying that status as a weighted score, which is then used to design and prescribe a individualized treatment plan (ITP). In some instances, an early part of the ITP involves intravenously injecting multipotent stem cells into the oral vasculature, allowing a period of time to pass, then re-assessing multiparameter values post-injection and re-scoring the patient's health and wellness status, noting improvements and developing another (second) ITP, which may include additional, periodic IV-injections to maintain and continue enhancing the patient's global, multisystem health and wellness.

In some aspects, provided herein is a dental procedure for improving general systemic health and overall wellness in a patient, comprising:

(a) establishing a pre-injection baseline periodontal status of the patient by measuring at least two parameters selected from: (i) patient's age; (ii) tooth pocket depth; (iii) number and position of bleeding points upon probing; (iv) intraoral camera photos recording level of gum tissue inflammation; (v) digital radiogram full mouth series (FMX) for facial/oral/dental bone levels/density; (vi) sample of blood, saliva and/or gingival fluid for analysis of at least one of:
1) periodontal pathogens and/or bacterial load (e.g., OralDNA testing);
2) levels of cytokines (e.g., TNF, IL-1, IL-6);
3) levels of immune and/or epithelial cells; and
4) expression levels of biomarkers; and

(b) according to the measures obtained, using a clinical calculator/nomogram to assess the patient's current status and prescribe an individualized treatment plan (ITP); and (c) intravenously injecting a composition comprising multipotent stem cells into the patient's oral vasculature; thereby increasing total systemic health and wellness.

In some embodiments, the method or dental procedure further comprises performing regular (e.g., every 30 to 90 days) periodontal cleaning and debridement in the patient.

In some embodiments, the method or dental procedure further comprises: (a) allowing approximately 90 days to six weeks to pass after the IV injection, then obtaining a post-injection reading of the parameters selected in baseline reading; and (b) making a first comparison of the pre-injection baseline reading to the post-injection reading; and, optionally, making a second comparison of both pre-injection and post-injection readings for each parameter to an ideal value(s). See FIGS. 1-6.

In some embodiments, the method or dental procedure further comprises at least one of: (a) based on the first and/or second comparisons, prescribing a course of additional dental and/or medical treatments, wherein, if the patient follows the prescribed course, the patient achieves a state of wellness having post-injection readings closer to the ideal value(s) than were pre-injection readings; and (b) repeating IV injection of the composition comprising multipotent stem cells (e.g., every 90 days over a one-year period) until the patient has any (or a specifically desired) percent (%)-improvement over pre-injection baseline readings.

In some embodiments, the method or dental procedure further comprises: (a) monitoring the improvement in the patient, as demonstrated by one or more measures selected from: an increased level of anti-inflammatory cytokines; a decreased level of pro-inflammatory cytokines; bacterial load reduction; reduction in levels of antiphospholipid antibodies and/or anticardiolipin (aCL) antibodies detecting myocardiocytes, or other symptoms of APS; a reduction of anticardiolipin signaling; a decrease of blood pressure; and reduction in or halting progression of bone loss and/or evidence of bone regeneration; other quantitative measures of epithelial regeneration, or any positive changes in the post-injection Access Genetics/OralDNA parameters tested.

In some aspects, provided herein is a method of treating a condition selected from antiphospholipid syndrome (APS), periodontal disease, an autoimmune disease, arthritis, a chronic inflammatory disease, viral (e.g. coronaviral, COVID-19) or bacterial infection, migraines, cognitive impairment/dysfunction, dementia and Alzheimer's disease (AD), comprising: IV-injecting into oral vasculature a composition comprising multipotent stem cells in an amount effective to treat the APS, periodontal disease, autoimmune disease, chronic inflammatory disease, viral or bacterial infection, and/or AD. In some embodiments of the method, the condition affects at least a portion of the digestive tract (including the oral cavity). In some embodiments of the method, the condition is a chronic inflammatory disease, or bacterial and/or viral infection (e.g. COVID-19), and the condition is periodontal disease. In some embodiments, the virus is COVID-19. In some embodiments of the method, the condition results in bone degeneration/erosion around natural teeth or implants. In some embodiments of the method, the composition stimulates dental bone regeneration, as measured by 2-dimensional and/or 3-dimensional x-rays and/or a decrease in pocket depth (indicating gingival attachment level change).

In some embodiments, the composition reduces bacterial infection. In some embodiments, the bacterial infection is mediated by Porphyromonas gingivalis (P.g.). In some aspects, the dental procedure or the method of treating the condition further comprises co-administering a small molecule inhibitor of P.g. In some aspects, disclosed herein is a composition for oral IV injection, comprising (or consisting essentially of) multipotent umbilical stem cells and a small-molecule inhibitor of P.g.

In some aspects, the dental procedure and/or the method of treating the condition comprises, along with the stem cells, co-administering autologous plasma rich platelets (PRPs).

In some aspects of the dental procedure and/or the method of treating the condition: the IV injection treats an early symptom of AD and/or dementia; the early symptom being treated is selected from cognitive dysfunction, memory loss, difficulty performing familiar tasks, difficulty planning, making decisions and/or solving problems, vision impairment, difficulty speaking, or a combination thereof; and the treatment is observed as an at least 20% improvement in a post-injection reading over a pre-injection baseline reading and/or a pre-treatment parameter.

In some embodiments, the dental procedure comprises: recording and storing the pre-injection baseline levels/readings of selected parameters (in some cases the parameters include markers specific to APS, such as measuring antiphospholipid and/or anticardiolipin antibody levels) using a 2-D or 3-D chart/nomogram for assessment (exemplified in Table 1 below, and FIG. 1, respectively) to score/quantify a health and wellness state; and/or comparing the score to ideal values stored on a computer readable format; allowing approximately 2, 3, 4, 5, 10, 14, 21, 28, 35, 45, 60 or 90 days to pass, or allowing between two and six weeks to pass post-injection, then measuring post-injection levels/readings of the selected parameters; retrieving pre-injection baseline readings and/or ideal values from the computer readable format; comparing post-injection levels/readings to the pre-injection baseline readings and ideal values; and using the comparison and quantification arrived at by using the 2-D or 3-D chart/nomogram (See Table 1 below, and FIG. 1, respectively) to prescribe a course/series of additional dental and/or medical treatments, called an ITP (and optionally a second, third, fourth and/or fifth ITP), prescribing a second ITP whereby the patient who follows the prescribed second ITP achieves specific subgoals over the course/series of ITP(s), progressing toward the ideal values until an at least 50% improvement is observed when post-injection readings are compared to the pre-injection baseline readings. Over the course of the ITP, the patient progresses, stepwise, such that, when the patient follows the prescribed ITP and full course of treatments, the patient achieves specific subgoals over the course/series, progressing toward the ideal values/goal of an at least 25% or 50% improvement is observed in post-injection levels/readings when post-injection readings are compared to the pre-injection baseline levels/readings and an overall improvement in systemic health and overall wellness is observed. In some cases, the comparison is used to develop and prescribe a second ITP, whereby the patient who follows the prescribed second ITP achieves a state of wellness having post-injection readings closer to the ideal value(s) than pre-injection readings achieves specific subgoals over the course of the ITP, progressing toward the ideal values until an at least 50% improvement is observed when post-injection readings are compared to the pre-injection baseline readings.

In some aspects, disclosed herein is a method of reducing the risk of occurrence of AD in a patient having an increased risk of AD, comprising IV-injecting into oral vasculature of the patient a composition comprising (or consisting essentially of) multipotent stem cells in an amount effective to reduce the risk of occurrence of AD in the patient, wherein the reduction of risk is observed when the patient achieves an age of at least 70 years of age or older without a significant loss in cognitive function, speech, vision, memory, ability to perform familiar tasks, ability to plan, make decisions and/or solve problems, or any combination thereof. In some embodiments, a patient has a genetic high risk of occurrence of AD at 70 years of age or older, and the patient being treated with multipotent stem cells is currently younger than 70 years of age.

In some aspects, disclosed herein is a method of treating migraine in a human patient, comprising: IV-injecting into oral vasculature a composition comprising (or consisting essentially of) multipotent stem cells (e.g., MUSCs) (without the need for pre-culturing, ex vivo expansion, reprogramming, transformation, immortalization, or inducing the stem cells (SCs) to stimulate and/or activate the cells prior to injection) in an amount effective to treat migraine in the human patient, wherein effective treatment is observed as a decrease in or cessation of migraine symptoms (e.g., pain, vision and/or aura issues).

In some aspects, disclosed herein is a method of treating joint pain, arthritis and/or fibromyalgia in a human patient, comprising: IV-injecting into oral vasculature a composition comprising (or consisting essentially of) multipotent stem cells in an amount effective to treat joint pain, arthritis and/or fibromyalgia in the human patient, observed as a decrease in pain or malaise symptoms experienced by the patient.

In some aspects, a method of treating joint pain, arthritis and/or fibromyalgia in a human patient is provided, comprising: IV-injecting into oral vasculature a composition comprising (or consisting essentially of) MUSCs in an amount effective to treat joint pain, arthritis and/or fibromyalgia in the human patient, observed as an increase in range of motion, and/or a decrease in pain and malaise symptoms experienced by the patient.

In some aspects, provided herein is a method of treating discomfort and/or pain related to inflammation of the digestive tract especially in relation to Fn (Fusobacterioum nucleatum) in a patient comprising: IV-injecting into oral vasculature a composition comprising (or consisting essentially of) multipotent stem cells in an amount effective to reduce inflammation of the digestive tract; ease disharmony in the stomach and GI tract, including acid reflux, which can also create wear on the teeth and can therefore be visualized, treat irritable bowel syndrome (IBS), clean up a “leaky gut” as observed by a decrease in the discomfort and or a decrease in pathogen burden; or treat diseases or disorders such as autoimmune connective tissue diseases, undifferentiated connective tissue disease (UCTD), chronic inflammatory disease, diabetes, viral or bacterial infection (e.g. COVID-19), APS, rheumatoid arthritis (RA), migraines, dementia, cognitive impairments, Alzheimer's disease (AD), Sjögren's syndrome, Myositis, Scleroderma, and SLE.

In some aspects, provided herein is a method of stabilization of insulin/sugar levels in a diabetic patient comprising: IV-injecting into oral vasculature a composition comprising (or consisting essentially of) multipotent stem cells in an amount effective to improve/maintain consistent insulin and/or sugar levels in the patient, observed as a decrease in A1C and/or at-home testing levels experienced by the patient.

### BRIEF DESCRIPTION OF THE DRAWINGS

The following detailed description is to be read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: presents a three-dimensional (“3-D” or “3D”) clinical calculator for assessing, and assigning a patient's health and wellness score (pre- and post-injection). The score is then used to design and prescribe an ITP.

FIG. 2: lists several exemplary parameters that can be assessed by Ayass Bioscience LLC for input into the clinical calculator for development of the ITP.

FIG. 3: presents test results from a particular patient in which several parameters were measured before injection, and used to develop and prescribe an ITP.

FIG. 4: presents exemplary results for a patient using OralDNA's MyPerioPath testing.

FIG. 5: presents actual OralDNA test results before treatment with stem cells by oral injection, as described below in Case Study 4.

FIG. 6: presents actual OralDNA test results 6 weeks after oral injection of stem cells in the same patient of Case Study 4.

### DETAILED DESCRIPTION

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

The global wellness model used in the systems and methods disclosed herein is based on assessment-driven therapy, including improving periodontal health, which has been observed to reduce the incidence and severity of many acute or chronic systemic diseases, involving immune and/or inflammatory responses, as well as those with a pathogen etiology or progression. Examples of such include, but are not limited to: cardiovascular disease (CVD), high blood pressure), endocarditis, migraine, plaque-based neurodegeneration (Alzheimer's, dementia), diabetes (insulin dependent diabetes mellitus (IDDM), pre-diabetes, insulin resistance, metabolic syndrome), autoimmune-based, and arthritic diseases.

Briefly, chronic periodontitis is a progressive inflammatory condition characterized as the degeneration of the supportive tissues surrounding teeth, and involving the resorption of bone, the laxation of ligament that attach teeth within bone, along with associated swelling, bleeding and localized necrosis of the gingival epithelium. The inciting cause of chronic periodontitis is largely believed to be a polymicrobial infection within the space between the gingiva and the teeth. Increased levels of selected pathogenic bacteria evoke inflammation of the periodontal tissues that lead to the degeneration of bone, ligaments and epithelium. The method and system described herein is a novel approach to the treatment of periodontal disease, that not only reduces the level of the bacteria that are a cause, but promotes healing and regeneration of these tissues.

Moreover, until the present disclosure, the relationship between such conditions and a patient's overall oral and systemic global multisystem health and wellness has not been considered and quantified for identification of subjects at abnormally high risk of developing an illness and likely to be a good candidate for therapeutic treatment by oral IV-injection with multipotent stem cells. Thus, there is value and need for a test that provides standard subject-identification criteria and that leads to a consistent diagnosis throughout childhood and into adulthood. The presently disclosed assays and system of developing and prescribing an ITP provides a clear and useful design that accommodates all age groups, races and many oral and correlated systemic diseases.

It has been hypothesized that some of these manifestations of disease are in part a consequence of the interaction of autoantibodies, including β2-glycoprotein 1 (β2GP1), the target antigen of anticardiolipin antibodies (aCl), with the endothelium, leading to vascular inflammation and thrombosis. Schenkein, et al. report that aCl from periodontitis patients can interact directly with endothelial cells to upregulate adhesion molecules and induce production of cytokines such as MCP-1 and some tissue factors by endothelial cells. (Schenkein, et al., J. Clin. Periodontol., 2013, 40(3):212-217. doi:10.1111/jcpe.12043).

Previous studies have indicated that a significant proportion of individuals with periodontitis demonstrate elevated serum concentrations of aCl (Schenkein et al., 2003, J. Dent. Res. 82(11):919-922). It has recently been demonstrated that oral bacterial pathogens may be cross-reactive with β2GP1, the target antigen of aCl (Wang et al., 2008) and that periodontal therapy can decrease systemic antibody levels of aCl (Gunupati et al., 2011). The observation that periodontitis patients' sera can contain elevated concentrations of aCl (Schenkein et al., 2003), and studies indicating that aCl can be associated with cardiovascular diseases even in patients without autoimmune disease (Artenjak et al., 2012) raise the question of whether the aCl found in periodontitis has pathogenic properties.

The Bale-Doneen method and compositions for predicting cardiovascular events are disclosed in US Patent Application publication 2012129708A1. Also described are methods, systems, devices, panels, and software for determining values for two or more markers in order to characterize a subject's risk of developing cardiovascular disease or experiencing a complication thereof (e.g., within the ensuing one to three years), and for identifying subjects in need of preventative therapy (e.g., statins). In certain embodiments, the markers are selected from: hs-CRP, ACR, Lp-Pla2, MPO, fibrinogen, KIF6, and F2 isoprostanes.

In a recent study, patients with generalized aggressive and chronic periodontitis as well as subjects without periodontitis but otherwise systemically healthy (as determined by medical history) received a comprehensive periodontal evaluation that included determination of pocket depth (PD), attachment loss (AL), plaque index (PI), and gingival index (GI) at 6 sites per tooth. At the time of examination a blood sample was taken and processed for serum which was stored at −70° C. Serum samples chosen for inclusion in this study were selected from those previously assayed for aCl content so as to provide sufficient numbers of sera within each diagnostic category to test hypotheses regarding the impact of diagnosis on aCl function. Sera testing positive for aCl from periodontally healthy subjects was limited due to the low prevalence of positive tests within the small group. The diagnostic criteria were as follows: Subjects with normal periodontium (NP) included those of any age with no evidence of AL or pockets greater than 3 mm, other than in buccal or lingual areas of gingival recession. Subjects with Chronic Periodontitis (CP) were of age >25 years with AL 2 mm or greater on more than one tooth in any extent or severity pattern that is consistent with plaque level and age. The definition of Generalized Aggressive Periodontitis (GAgP) for the subjects in this report conform to current diagnostic guidelines for these diseases with the additional requirement that such patients had a history of disease onset prior to age 35. They presented with at least 8 teeth with 5 mm or more attachment loss at interproximal sites, at least 3 of the affected teeth were not first molars and incisors. The total initial sample number included 23 from subjects with CP, 15 samples from subjects with GAgP, and 21 NP subjects. (Schenkein, et al., J. Clin. Periodontol., 2013, 40(3):212-217. doi:10.1111/jcpe.12043).

Thus, assessment of endothelial function has been one approach that investigators have used to study associations of periodontitis with CVD and to assess the impact of periodontal therapy on CVD measures (Amar et al., 2003, Higashi et al., 2008, Pischon et al., 2007, Tonetti et al., 2007). These indirect approaches to assessment of CVD risk indicate that there is endothelial dysfunction associated with periodontitis that may be improved by therapy aimed at decreasing periodontal inflammation.

In some embodiments of the present disclosure, the multipotent stem cells used in the method of treatment comprise multipotent umbilical stem cells (MUSCs). In some embodiments, the multipotent stem cells are derived from the umbilical cord, Wharton's jelly, amniotic fluid, or from placental tissue. In some embodiments, the composition comprises a mixture of more than one type of stem cells. In some embodiments, the multipotent stem cells are living/viable. In some embodiments, the multipotent stem cells are not living/inviable. In some embodiments, the composition comprises an acellular commercial product. In some embodiments, the composition comprises Wharton's jelly. In some embodiments, the composition comprises amniotic fluid. The multipotent stem cells may be obtained from any source, including autologous stem cells from the patient to be treated, or may be obtained from a commercial source. One example of a commercial source of the multipotent stem cells is Invitrx Therapeutics, Lake Forest, Calif. In some embodiments, the multipotent stem cells do not require in vivo or ex vivo procedures to activate, expand, reprogram, transform, immortalize, or induce the SCs prior to injection/administration. In some embodiments of the method, the composition also includes plasma rich platelets (PRP).

In some cases, the administration comprises injecting increasing amounts of multipotent stem cells and/or over increasing treatment timeframes (depending upon the persistence and/or severity of chronic disease).

In some embodiments, improvement is observed in the patient as a self-reported reduction in a symptom (e.g., pain, cognitive function, memory loss, etc.), or is demonstrated by an improvement in one or more measures selected from: an increased level of anti-inflammatory cytokines; a decreased level of pro-inflammatory cytokines; bacterial load reduction; reduction in levels of a protein or DNA biomarker of disease, reduction in levels of antiphospholipid antibodies and/or other symptoms of APS; a reduction of anticardiolipin signaling; a decrease of blood pressure; and reduction in or halting progression of bone loss and/or evidence of bone regeneration; other quantitative measures of epithelial regeneration, or any positive changes in the post-injection Access Genetics/OralDNA parameters tested.

In some embodiments, the patient achieves a series of subgoals over the course of the ITP being implemented, and the patient is progressing toward a goal of at least 60% improvement in one or more post-injection readings as compared to the pre-injection baseline readings in the one or more of the selected parameters. In some embodiments, the improvement is the observance of now-stable tooth pockets with no bleeding. In some embodiments, the improvement is a reduction of oral inflammation. In some embodiments, the improvement is a reduction in one or more biomarkers and/or symptoms of inflammation. In some embodiments, the improvement is a reduction in one or more biomarkers and/or symptoms of bacterial or viral infection (e.g. COVID-19). In some embodiments, the improvement is a reduction in one or more biomarkers and/or symptoms of metabolic disease, diabetes or pre-diabetes. In some embodiments, the improvement is a reduction in one or more biomarkers and/or symptoms of atherosclerosis and/or cardiovascular disease. In some embodiments, the improvement is observed as bone regeneration. In some embodiments, the improvement is a reduction in one or more biomarkers and/or symptoms of Alzheimer's disease. In some embodiments, the improvement is a reduction in one or more biomarkers and/or symptoms of cognitive impairment. In some embodiments, the improvement is a reduction in patient-reported pain and/or the occurrence or severity of migraines.

In some embodiments of the method, provided herein is the use of IV-injected MUSCs as well as an associated protocol to assay (pre- and post-injection) a variety of parameters including assessing, from a sample of the patient's bodily fluid (blood, saliva, mucus, urine, etc.) taken from the oral cavity (e.g., near the site of injection) and/or periphery (such as systemic circulation): the presence of and/or levels of pathogenic bacteria and other infectious agents that cause periodontal inflammation, a series of host specific protein or nucleic acid (DNA, RNA) biomarkers, bone density, periodontitis, dental caries, atherosclerosis, cardiovascular disease, cholesterol levels, diabetes (glucose, insulin, hemoglobin A1c). After treatment and the patient's adoption of the prescribed ITP, a reduction in the clinical signs of inflammation, evidence of new bone growth/reduced bone loss, reduced pocket depth, reduction of point bleeding and (other objective) signs of tissue regeneration are observed. Improvement of periodontal health is associated with lowering of the incidence and severity of systemic disease, such as atherosclerotic cardiovascular disease (ASCVD), plaque-based neurodegeneration, insulin dependent diabetes mellitus and various arthridites. This present disclosure also describes an association of periodontal disease with several systemic diseases (with or without the coincidence of the presence of aCL or antiphospholipid antibodies), which is used in the assessment of a patient's current health and wellness status and risk of developing future illness or particular diseases, and provision of an ITP. The present disclosure describes the therapeutic use of MUSC (via the system and methods described herein) and their efficacy in improving the symptoms of, or even preventing, several oral and systemic diseases.

In some embodiments of the method, the subject is a mammal. In some embodiments of the method, the mammal is a human. In some embodiments, the method further comprises the step of genotyping the subject for the presence of at least one risk allele. In some embodiments of the method, the subject has been diagnosed as having at least one risk factor or allele associated with development and/or progression of atherosclerosis. It has been proposed that the development and progression of many systemic disease involves at least two “hits,” occurring in no particular order. One hit may be the development of periodontal disease, dental caries or oral inflammation and/or infection. Another hit may be the incidence of or an increase in circulating antiphospholipid antibodies (aPLs) and endothelial system dysfunction. Another hit may involve complement activation, the complement cascade and/or coagulation, often marked by up-regulation βI2GP1 receptors on endothelial cells triggered by inflammation (infection, trauma, surgery) (Corban, et al., 2017, J. Am. Coll. Cardiol. 69(18):2317-2330). The oral IV-administration of multipotent stem cells according to the presently described methods decreases the severity of the symptoms, reverses one or more of the “hits,” reverses the systemic disease patterns, and/or delays their onset.

Periodontal disease or dental infection often coincides with extreme gut disharmony (e.g., “leaky gut,” gluten sensitivity, IBS, colitis, and other digestive system disorders involving an immune and/or inflammatory response), APS, lupus, Sjögren's syndrome, and connective tissue diseases. A patient's APS may be undetected until the patient is tested at the commencement of the system and methods described herein. Due to the toxic nature of the bacteria involved in periodontitis, induction of the complement carcase and/or appearance of or acceleration of systemic disease (e.g., APS) often occurs and causes additional triggers, and increases endothelial degradation or induction of other diseases such as cardiovascular disease, autoimmune disease, diabetes, atherosclerosis, or Alzheimer's disease for example). Oral IV-administration of multipotent stem cells leads to a decrease in (or even elimination of) the infectious agent, healing of endothelial tissue and downregulation/quieting the receptors involved in infection and/or inflammation. Addition of traditional medical and dental therapies (e.g., deeper cleanings, co-administration of blood thinners, statins, anti-inflammatory agents, immunosuppressant agents, etc.) serve to enhance the improvements seen in the patient.

Provided herein are methods of using multipotent stem cells in treatment or prophylaxis of disease, in a system for improving general health and overall wellness. The disclosure provides a method in which multipotent stem cells are intravenously administered within the oral cavity, which has a rapid effect on symptoms of inflammation and/or infection, not only treating periodontal diseases, but also reducing bacterial load, pain, bone loss, etc. as well as stimulating new bone growth and achieving other hallmarks of improvement in health and overall systemic wellness. In some instances, a sample of a bodily fluid is drawn from the patient before injection to measure baseline levels of several biomarkers to obtain the patient's status on the spectrum of health and wellness and/or to compare post-treatment effects of the injected stem cells, to measure improvement, and to develop a treatment plan for enhancing multisystem wellness.

As disclosed in US Patent Publication 2017/0199189, herein incorporated by reference in its entirety, a linkage has been identified between a patient's oral health or periodontal disease and the patient's systemic health or systemic disease status (e.g., autoimmune connective tissue diseases, APS, RA/rheumatoid arthritis, Sjögren's syndrome, Myositis, Scleroderma, lupus (SLE), undifferentiated connective tissue disease (UCTD)). autoimmune disease, chronic inflammatory disease, viral or bacterial infection (e.g. coronaviral, COVID-19), diabetes, migraines, cognitive impairments, and Alzheimer's disease (AD). This oral-systemic health linkage is measurable and quantifiable using the methods described herein, and an individualized treatment plan (ITP) can be prescribed, including one or a series of intravenous, intraoral injections of multipotent stem cells as a part of a system of achieving improved and/or enhanced global/multisystem health and wellness.

Porphyromonas gingivalis (P.g.) is a Gram-negative oral anaerobe that is involved in the pathogenesis of periodontitis, an inflammatory disease that destroys the tissues supporting the tooth, eventually leading to tooth loss. Pg is an opportunistic oral pathogen, and can locally invade periodontal tissues and evade the host defense mechanisms. In doing so, it utilizes a panel of virulence factors (such as cysteine proteinases (gingipains), lipopolysaccharide (LPS), capsular polysaccharide (CPS) and fimbriae) that cause deregulation of the innate immune and inflammatory responses. (Bostanci and Belibasakis, 2012, FEMS Microbiol Lett 333:1-9).

Interestingly, new evidence regarding the cause of Alzheimer's disease has been reported. Compelling evidence that the condition is caused by a bacterium involved in gum disease could prove to be important in tackling one of medicine's biggest mysteries, and lead to effective treatments or even a vaccine. Multiple teams have been researching Porphyromonas gingivalis, a bacterium involved in gum disease, as risk factor for Alzheimer's. P.g. actively invades and inflames brain regions affected by Alzheimer's; gum infections can worsen symptoms in mice genetically engineered to have Alzheimer's; and it can cause Alzheimer's-like brain inflammation, neural damage and amyloid plaques in healthy mice. P. gingivalis appears to use two toxic enzymes to feed on human tissue in 99 and 96 percent of 54 human Alzheimer's brain samples taken from the hippocampus (involved in memory). These protein-degrading enzymes are called gingipains, and they co-localize with plaques found in higher levels in brain tissue that also had more tau fragments and thus more cognitive decline.

A bacterial hypothesis for Alzheimer's doesn't conflict with genetic evidence. The human body's propensity for inflammation can vary according to genetic variations that affect our immune systems, and this may influence how much damage P. gingivalis induces in a brain. The biggest genetic risk factor for Alzheimer's is a variant of the gene that makes the ApoE immune protein. Recently, a team in Sweden found that the gingipains released by P. gingivalis break up the ApoE protein into fragments, cleaving it at the site of a particular amino acid within the protein, and that these fragments may harm nerves. The ApoE4 variant of this protein contains more of this amino acid, suggesting that the reason people who make this variant are at a higher risk of developing Alzheimer's may be because harmful levels of ApoE protein fragments build up more quickly in their brains than in those of other people. (Dominy, et al., 2019, Sci. Adv. 5:eaau3333; Deborah MacKenzie, 2019, Science Advances, issue 3215 [Online at newscientist.com/article/2191814-we-may-finally-know-what-causes-alzheimers-and-how-to-stop-itMixzz5ux2GSntY]).

Antiphospholipid syndrome (APS), sometimes known as Hughes syndrome, is a disorder of the immune system that causes an increased risk of blood clots. This means people with APS are at greater risk of developing conditions such as: deep vein thrombosis (DVT), a blood clot that usually develops in the leg; arterial thrombosis (a clot in an artery), which can cause a stroke or heart attack; and blood clots in the brain, leading to problems with balance, mobility, vision, speech and memory. Pregnant women with APS also have an increased risk of having a miscarriage, although the exact reasons for this are uncertain. APS doesn't always cause noticeable problems, but some people have general symptoms that can be similar to those of multiple sclerosis.

The antimalarial drug hydroxychloroquine (Plaquenil®), statins (cholesterol-lowering agents), B cell (a type of white blood cell) inhibition, and complement inhibition have recently been studied systematically in aPL-positive patients. In addition, recent findings suggest that mechanistic target of rapamycin (mTOR) pathway activation plays a role in the blood vessel inner layer thickening in aPL-positive patients, which leads to blood clot formation in small blood vessels. (Sirolimus, also known as rapamycin, is a medication that blocks mTOR pathway activation, which is approved for other diseases). Hydroxychloroquine (Plaquenil) is an antimalarial medication, which is used to treat systemic lupus erythematosus (SLE, also known as lupus). Hydroxychloroquine has anti-inflammatory effects and also inhibits platelet clumping, which is a key step in blood clot formation. There is evidence to suggest this drug may help reduce the blood clotting effects of aPL in mice and also decrease the risk of blood clots in SLE patients. In order to understand the protective role of hydroxychloroquine in aPL-positive patients without other systemic autoimmune diseases (for instance, lupus) controlled studies are planned or undergoing. For the time being, hydroxychloroquine may be used as an adjunctive therapy (supportive treatment used together with the primary treatment) in APS patients with difficult-to-control aPL-related clinical problems.

Additionally, studies have demonstrated associations of periodontitis with conditions including APS and/or cardiovascular disease (CVD) and stroke. A fundamental pathologic characteristic of CVD is upregulation of the endothelium promoting egress of inflammatory cells from the bloodstream and into atheromatous lesions (Libby et al., 2009). Furthermore, the presence of aCl is also associated with CVD even in patients without autoimmune disease, raising the question of whether the aCl found in periodontitis itself has pathogenic properties. To this point, oral bacterial pathogens have been reported to cross-react with β2-glycoprotein 1 (β2GP1), the target antigen of aCl. Similarly, a variety of microbial pathogens have been reported to induce the production of cross-reactive aCl-like antibodies that can induce APS-like symptoms in animal models, leading to the hypothesis that some infectious diseases can promote APS-like symptoms, including accelerated atherosclerotic CVD, in the absence of autoimmune disease. (Schenkein, et al., J. Clin. Periodontol., 2013, 40(3):212-217. doi:10.1111/jcpe.12043).

The compositions, methods and specific system presented herein are based on this oral-systemic health linkage. The present disclosure describes and treats the association of periodontal disease with co-occurring systemic disease(s); thus, to achieve more global wellness (a.k.a. “systemic wellness,” “multiple system wellness,” or “multisystem wellness”), dental professionals now must consider treating the population of patients with acute infections (e.g. COVID-19) and/or underlying autoimmune diseases (e.g., APS, RA, Sjögren's syndrome, Myositis, Scleroderma, Lupus (SLE), undifferentiated connective tissue disease (UCTD)) starting in the oral cavity and oral vasculature.

As disclosed herein, treatment of systemic disease and improvement in global wellness and even life-expectancy starts in the top of the gastrointestinal tract, i.e., the oral cavity. As described herein, periodontal disease is often coincident with acute or chronic systemic diseases involving immune and/or inflammatory responses, as well as disease with a pathogen etiology or progression. Such coincidence is not due to chance, but rather, is directly and strongly correlated.

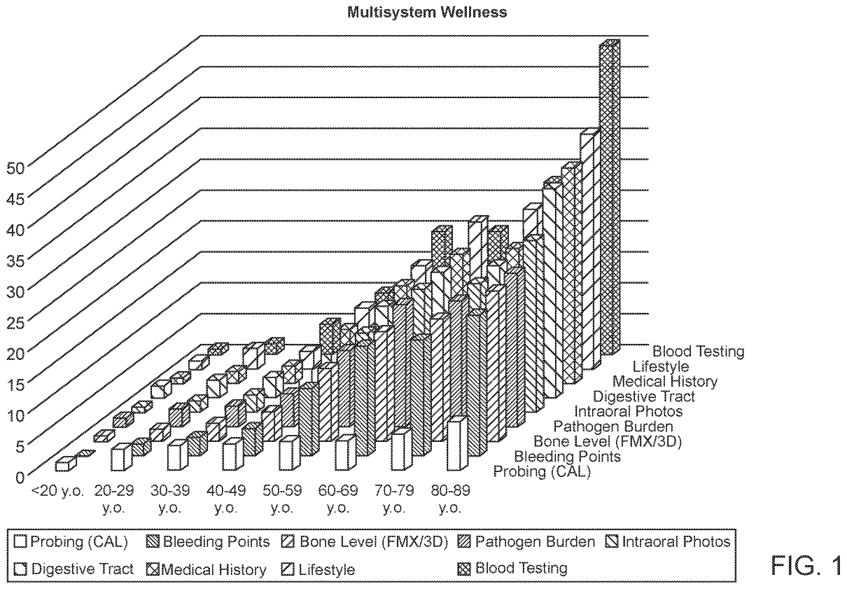

As used herein, a “clinical calculator”, “nomogram,” “chart,” “diagram” “matrix,” or “objective metric” refer to a tool such as those exemplified as Table 1 below, and FIG. 1. Theses clinical calculators represent the relations between three or more variable quantities by means of a number of scales, so arranged that the value of one variable can be found by a simple geometric construction, for example, by drawing a straight line intersecting the other scales at the appropriate values, allowing the classification and quantification of symptoms and identification of a patient candidate for treatment by the methods and system disclosed herein. Table 1 presents a two-dimensional (a.k.a. “2-D” or “2D”) clinical calculator (a.k.a. “chart,” “nomogram,” “matrix” or “metric”) for assessing/determining/assigning a patient's weighted score, which is then used to design and prescribe a individualized treatment plan (ITP).

TABLE 1

| | age (optional) | | | | | | | |
|---|---|---|---|---|---|---|---|---|
| | Column 1 | Column 2 | Column 3 | Column 4 | Column 5 | Column 6 | Column 7 | Column 8 |
| Parameter | <20 y.o. | 20-29 y.o. | 30-39 y.o. | 40-49 y.o. | 50-59 y.o. | 60-69 y.o. | 70 y.o. | 80 y.o. |
| Probing | All 1-2 | All 3-4 | Any > 4 | | Low Vit D | Osteoporosis/ | Migraines/ | |
| (CAL) | | | | | | bisphosphonates | TMJ/ | |
| | | | | | | | headaches | |
| Bleeding | Blood | None | 1 to 3 | 4 to 10 | 11 to 20+ | Skeletal & Joint | replacement | |
| Points | Thinner | | | | | Pain | joints | |
| Bone Level | Lamina Dura | 1 to 3 | 4 to 10 | 1 Wall | Isolated | Generalized | severe bone | |
| (FMX/3D) | Intact | | | Defects | Horizontal | Horizontal/ | loss/several | |
| | | | | | | Dental Infection/ | missing | |
| | | | | | | Arterial | teeth/multiple | |
| | | | | | | Calcifications | RCT implants | |
| Pathogen | None | Low only | Low & | Low & | High but | High Above/Fn | Pg/Apo-e 3/4 | Alzheimer's |
| Burden | | | Medium | Medium | below/Fn | | | disease |
| | | | | Above | | | | |
| Intraoral | None | Flat | Isolated | Ring 1-2 mm | Ring 3-4 mm | Generalized | auto-immune | |
| Camera | | | Papilla/ | | | Inflammation | White/Heart | |
| Photos | | | Lower | | | | Purple | |
| | | | lingual | | | | | |
| | | | posterior | | | | | |
| Digestive | Healthy | Wear/ | Acid Reflux | Gluten/food | Food | Asthma/Lung | colitis/ | |
| Tract | | Divots | | sensitivities/ | Allergies | Issues/Chronic | irritable bowel | |
| | | | | Leaky gut | (e.g. peanuts) | sinus issues | | |
| Medical | Healthy | Fatigue | HRT/ | metabolic | Pre-Diabetic | Diabetes | uncontrolled | Event: |
| History | | | Hormone | syndrome/ | | Controlled | diabetes | Heart Attack/ |
| | | | imbalance/ | insulin | | | | Stroke/ |
| | | | Thyroid | resistance | | | | TIA |
| | | | | | | | | COVID-19 |
| Lifestyle | stress mild/ | excess | Waist or neck | Stress | Exercise Rare | Sleep Issues/ | Stress High/ | Cancer |
| | exercise 3-4 | weight/ | circumference | Moderate | | Cpap | exercise never/ | |
| | x day, sleep | elevated | above normal | | | | extreme | |
| | good | BMI | range | | | | fatigue | |
| Blood Work | no hormone | increased | Genetics | Rx of BP, | Increased | Blood work (+) | auto-immune, | autoimmune |
| Testing | issues, | BP, hormone | Positive: Il-1, | cholesterol, | CIMT, stress | for cardio, | coagulation, | disease |
| | diabetes, | replacement | Il-6, TNF, | Statins, etc. | test, echo, | diabetes, | cardiolipin, | (methotrexate) |
| | auto-immune | therapy | Heart Attack | | ultrasound, | endocrine, | even with no | |
| | or heart | | Gene, etc. | | thermography | cytokines, | symptoms | |
| | disease | | | | | advanced | | |
| | | | | | | inflamm. markers | | |

*Age: As the patient ages (shown using increasing column number rightward from column 1, youth (<20)) toward column 8), and/or as the severity of risk factors increases, (moving down the table from top to bottom rows), the greater the concern. *Lightest grey (e.g., column 1): Currently Healthy Presentation. *Medium grey (e.g. column 4): Oral health needs improvement. Test & initiate inflammatory reduction protocol (both SCs & traditional therapy/retest & re-assess. *Darkest grey (e.g., column 7): Critical concerns warranting amelioration of symptoms, arrest of disease progression, and/or a plan to reduce risk of disease. (Outlying medical conditions assessed on per case basis).

As disclosed herein, a patient may be eligible for and/or determined to be a candidate for the method of treatment by administering oral IV-injected multipotent stem cell therapy when a patient presents with periodontal disease of ADA class 3, for example. Other criteria for inclusion can be, for example, a loss of 30% or more dental or maxillofacial bone on a digital radiogram or x-ray.

In some embodiments, the clinical calculators exemplified in Table 1 and the Figures may be used by a dental or medical professional according to the method herein disclosed. For example: Several rows are clinical/diagnostic in nature. The table(s)/nomogram(s) are given to a clinician, for evaluating a patient at the time of a dental visit. Not all parameters must be measured. Chairside assessments are made, such as assessing the number of bleeding points (shown in the second row of Table 1). (For example, a color or shading may be associated with each box, where, for example, no bleeding points might be indicated by green or light grey shading, 1-20 by yellow or moderate shading, and 20+ by red or dark shading). Alternatively or in addition, columns and/or rows may be numbered with increasing value from left to right and top to bottom to indicate increasing concern. In some instances, and the colors/boxes have a score associated with them, such that, for example, the less critical parameters are in the upper left of the table, and may be color-coded green (or lighter grey, using a monochromatic greyscale) and given a subscore of 1, while the most critical parameters are in the lower right of the table, and may be color-coded red (or darker grey, using a monochromatic greyscale) and given a subscore of 5. The scores of a patient's boxes/parameters are summed to arrive at a patient's total score. Other parameters can include greater than 4 mm probing depth (a.k.a. clinical attachment loss (CAL)). Generalized horizontal bone loss may be in the darker shaded or red zone (e.g. Third row, column 7 of Table 1). The assessments can include an OralDNA test, and determination of an additional score considered and accounted for in the patient's total score. Where an intraoral camera is used, the gums show intense generalized inflammation, for example, but if the patient only has one or two areas of bleeding points or greater than 4 mm probing death (CAL), the patient may have a low total score and a minor case of periodontal disease without great concern or urgency of a systemic issue in need of treatment by a simple one-time administration of oral, IV-injection of multipotent stem cells, and may not require a complicated regimen to be developed by the method and system disclosed herein.

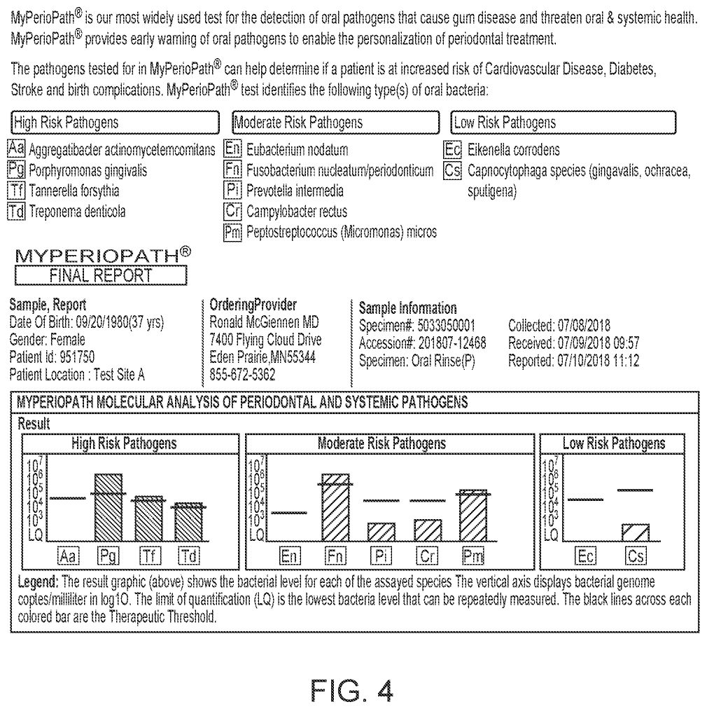

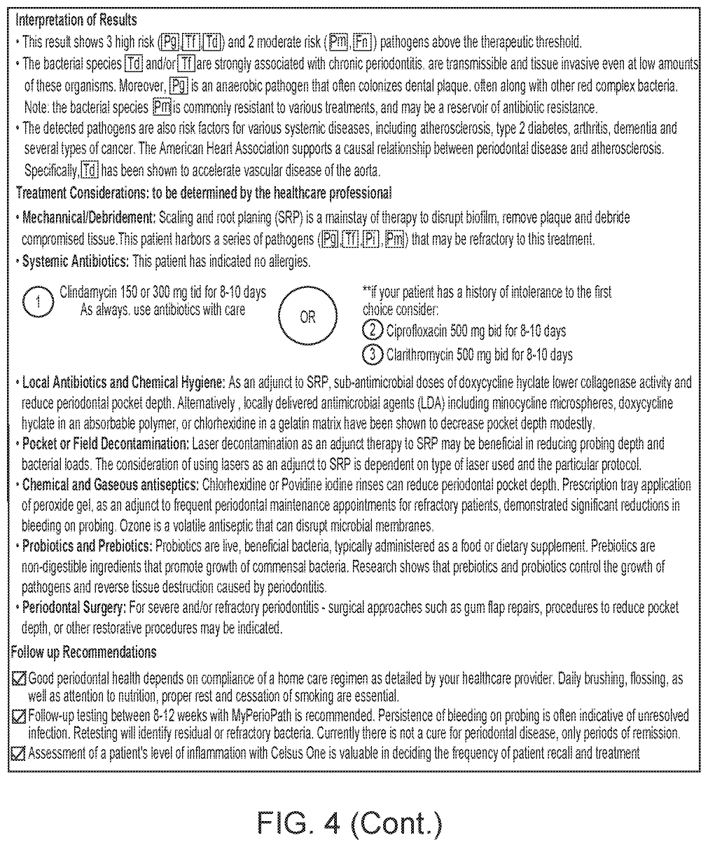

In another example, a 50 year old, having oral inflammation, low Vitamin D, joint pain, significant tooth mobility, decreased bone density, bisphosphsophonates, an APO 3/4 genotype, and P.g. found in OralDNA testing, greater than 9 mm pocket depth, and a medical history of high cholesterol and arterial calcifications may be considered at risk of future cardiovascular disease and may be considered for more urgent and immediate treatment by the methods and system disclosed herein. (See Table 1 and FIGS. 1-6).

In another case, if probing depth reveals greater than 4 mm pockets, the patients age can be less important, and low vitamin D becomes one of the differential diagnoses to consider, to determine an ITP including multipotent stem cell treatment to support bone health. Interim clinical assessments and the nomogram allows assessment of the patient's pre- and post-injection health and wellness status, as well as a means of measuring progress as a patient adopts the ITP.

The nomogram can be provided to a dental hygienist as a wipable vinyl or plastic sheet, and used as the beginning of a convenient assessment that feeds into the development and prescription of an ITP to a patient. After the nomogram-based assessment and scoring, on the dentist alone or in collaboration with another medical practitioner can arrive at a list of necessary next steps, testing, dental and/or medical procedures, medications, vitamins, hormones, and dietary recommendations. Another advantage of the presently disclosed method and system is that a patient may be aware of having high blood pressure, and may know of a family history of atherosclerosis, for example, and the additional parameters identified on the nomogram may suggest an ITP prescribing carotid intima testing; such additional testing as part of the ITP may reveal inflammation and hard and soft plaque formation indicating a more urgent state of active heart disease, of which he or she was not previously aware.

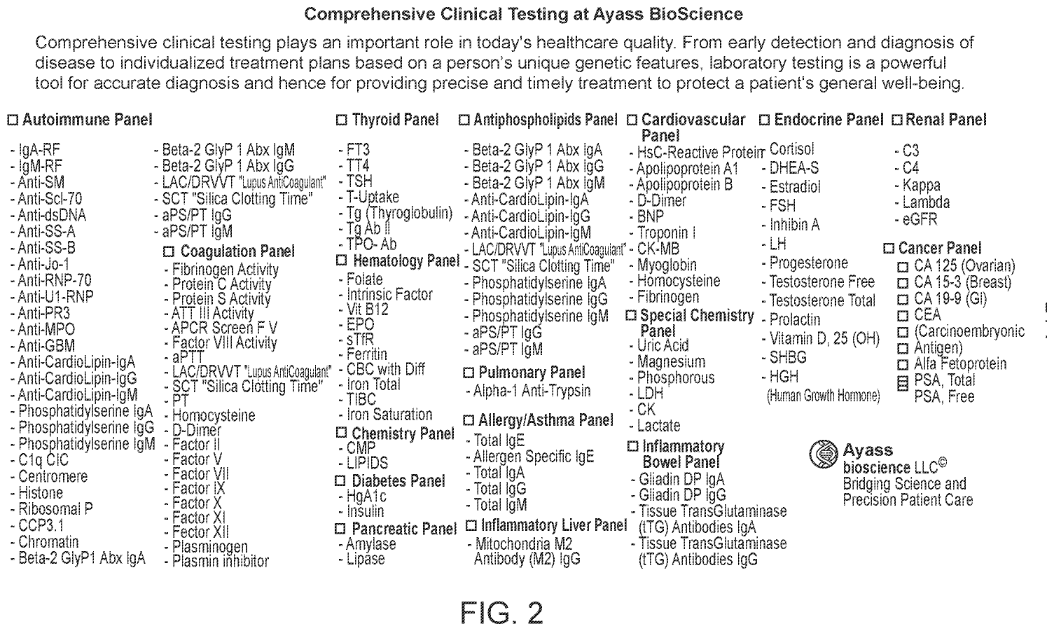

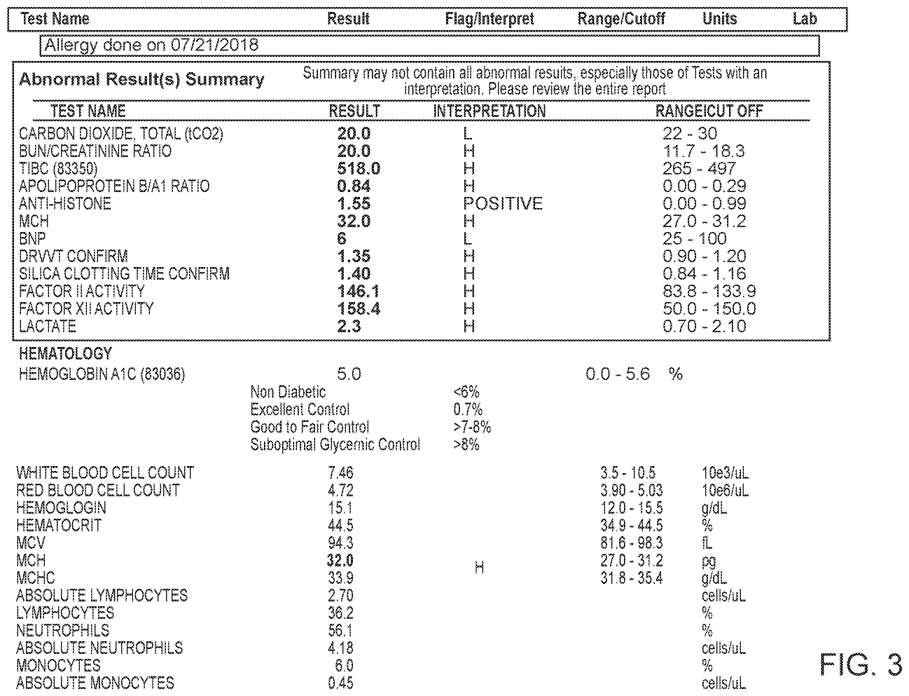

As disclosed herein, the methods and systems begin with assessment of parameters and treatment within the digestive tract, specifically as an IV injection within oral cavity. While not wishing to be held to any particular mechanistic theory, it is believed that the IV-injection of multipotent stem cells in oral vasculature allows the active agent more immediate access via anti-inflammatory factors, innate or adaptive immune system activation, etc., to brain vasculature (and possibly even through the blood-brain barrier (BBB)) in addition to oral and general peripheral circulation, allowing therapeutic effects to be observed much more rapidly than other therapies. The present disclosure is the first to conceive of such a method including assessment of health or disease using several parameters indicative of the oral-systemic link and allowing development and prescription of an ITP including one or more oral IV injections of multipotent stem cells. The method and system described herein have been observed to have nearly immediate effects on several parameters such as fighting inflammation, reducing bacterial infection, treating periodontal disease, and may ultimately result in stimulation of dental bone regeneration. The method and system described herein allow a far less subjective assessment of a patient's overall health and wellness. (See Examples and Case Studies 1 through 5, below). Even food allergies can be tested and analyzed with Ayass testing (FIG. 2).

A patient's medical history and current symptoms can be divided into treatment-related categories such as, but not limited to: 1) hormones, 2) nutrition including GI tract & diabetes, pre-diabetes, or metabolic disorder. 3) neurological system (including CNS and PNS, pain, and possible interactions with skeletal boney features; 4) inflammatory and immune system, cardiovascular diseases, infections and toxins. Such initial categorization may allow a more systematic placement of a patient into the nomogram assessment.

In some embodiments of the method, a test is performed to measure the level of stem cell engraftment, providing a quantitative assessment of whether a stem cell graft “takes” and over time what proportion of the cells at the site of injection, or elsewhere, are from the stem cell injection or from host.

The multipotent stem cell therapy described herein equips and signals the body's natural ability to activate reparative mechanism. By optimizing the cellular environment inside the body natural healing can begin, starting in the oral cavity. The multipotent stem cells IV-injected into the oral vasculature can naturally reverse the inflammatory conditions causing pain and damage in a patient's body, and can stimulate diseased cells and tissues to heal and regenerate.

Early phase periodontal disease is not painful and can go unidentified for many years until bone destruction is severe. As in diagnosis of diabetes and heart disease, there is no reason to wait until serious and debilitating symptoms have occurred. Periodontal disease can be identified earlier and treated therapeutically in order to decrease the overall burden on the body's environment. Un-programmed multipotent stem cells can help seek out and address the root causes of inflammation and support treatment of periodontal disease, resolve bacterial infections, reduce bacterial load, and encourage regeneration and repair. Decreasing inflammation is important in supporting healthier stronger living.

Pain Relief: (joint (TMJ/TMD) pain, migraines, pain from dental infections, pain from dental surgical procedures). Dental pain and migraines can be excruciating with little to no relief until strong opioid medications are used. In light of the opioid epidemic, alternative non-addictive solutions to pain are sorely needed. The method and system described herein provides a natural solution to subside or relieve pain quickly. In fact, there have been some recent advances in using stem cell therapy for chronic pain management. (Chakravarthy, et al., 2017, Pain Physician 20:293-305).

Mesenchymal stem cell therapy properties can effectively reduce inflammation and pain quickly. They have been utilized in orthopedics to assist in joint specific issues. Dentistry includes assessment of Temperomandibular Joint (TMJ) pain and treatment of with the and the pain associated with it, involving the connected musculature, associated cartilage, and frequent migraine symptoms. These debilitating symptoms have been rapidly reduced after a simple injection into the vasculature of the oral cavity in affected patients. Similarly, the pain of arthritic has been reported by some patients to become more tolerable with a noticeable increase in joint flexibility.

Presented herein is a scientifically researched and proven methodology that results in true periodontal regeneration utilizing stem cell therapy accelerating new bone growth, gum tissue reattachment, and reduction of pathogens while reducing pain. The method begins at the dental office, as a routine visit, but with the addition of taking a sample of saliva, blood or other bodily fluids and testing using companies such as Access Genetics and/or OralDNA Labs, and then employs a nomogram to assess systemic involvement and to develop and ITP.

Umbilical cord blood-derived hematopoietic progenitor cells are blood-forming stem cells, and a commercial sample of cord blood-derived multipotent stem cells may or may not contain a small, but significant amount of mesenchymal stem cells which also signal self-healing and regeneration. These multipotent stromal (connective tissue) cells stimulates the body to cause differentiation of the stem cells into a variety of cell types, including neurons, cartilage, fat, blood, bone, and muscle. Because multipotent stem cells can develop into multiple types of cells, but divide a limited number of times, this provides a built in safety mechanism such that there are few concerns about long term negative effects of the treatment. They are messengers that signal the body to activate self-healing and regeneration. The cells themselves do not create new tissue or healing, they signal the body to activate a cascade of cellular healing functions.

If a patient's health is deteriorating and he/she is noticing accelerated aging that patient should be considered as a potential candidate for treatment using the method and system described herein employing oral IV-injected multipotent stem cells, which encourages self-repair and healing. A simple oral IV-injection, which feels similar to the numbing used for a filling is all the patient experiences, and the entire procedure takes less than 10 mins. Frozen mesenchymal cells are thawed and injected without dilution. Thus, administration is quick and downtime is limited to the period of numbness that wears off quickly. Mild discomfort at injection site might be noticed for 24 hrs.

The present disclosure involves the combined effects of diagnostic testing to assess disease severity, to reduce bacterial levels, to promote healing and tissue regeneration through the application of multipotent stem cell injections into the oral vasculature. The treatment has been clinically demonstrated to result in new bone growth within the periodontium, reduce CAL and associated periodontal pocket depths, markedly reduce clinical and laboratory signs of acute and chronic inflammation, provide sustained reduction in periodontal pathogen levels, decrease symptoms of oral, head and neck and periodontal pain, and slow disease progression.

The developing science of stem cells has succeeded in regenerating parts of the heart, muscles, bone and nervous system. Such developments have also included oral and dental tissues. For example, stem cells have been used for regeneration of the periodontium, alveolar bone, dentine-pulp complex, craniofacial bone, mucosal tissue, tongue muscle, and for returning the function of salivary glands. (Bakhtiar, et al., (2018) Progress in Biomaterials 7:249-268).

Mesenchymal stromal cells (MSCs) have stem/progenitor properties, possessing broad immunoregulatory properties. Their clinical potential and the dynamic interplay between MSCs and the innate and adaptive immune systems is not yet understood. MSCs may sense and control inflammation, highlighting the central role of macrophage polarization. MSCs were reported to have potential clinical application in treating immune-based disorders. However, certain functional differences have been observed between in vivo and in vitro contexts and between species (as is the case between humans and mice, where MSCs are intrinsically different). The application of MSCs to treat Graft-versus-Host Disease (GvHD) has been studied in mice (Bernardo and Fibbe, 2013, Cell Stem Cell 13:392-402).

In dentistry, adult mesenchymal stem/stromal cells (MSCs) have been identified in oral and maxillofacial tissues, suggesting that the oral tissues are a rich source of stem cells, and were predicted to be useful as a source for genetically reprogrammed induced pluripotent stem (iPS) cells. Several studies have stirred interest in stem-cell-based therapies, tissue replacement therapies, tissue engineering approaches, and chair-side cellular grafting approaches using autologous MSCs. (Egusa, et al., 2012, J. Prosthodontic Research, 56:151-165, Part I; Egusa, et al., 2012, J. Prosthodontic Research, 56:229-248, Part II)

Embryonic stem cells (ESCs) and adult stem cells are the two main types of stem cells for tooth regeneration. Despite high proliferation and differentiation capabilities, ESCs are rarely applied in clinical practice because of possible tumorigenesis and ethical issues. Thus, recent studies on seed cells for tooth regeneration have mainly focused on adult stem cells. Among the adult stem cells, dental stem cells have been considered as a candidate for tooth regeneration. These include the dental pulp stem cells, stem cells from exfoliated deciduous teeth, periodontal ligament stemcells, stemcells from apical papilla, and dental follicle progenitor cells. All of these cells have already been proved owning multipotent and odontogenic differentiation potentials, and some of them have successfully applied into tooth regeneration studies. However, the use of dental stem cells has several potential limitations. The primary challenge is the limited availability of dental stem cells, especially from those who are agomphious. In addition, cellular rejection and ethical issues in allogeneic therapy further hinder the clinical application of dental stem cells. (Chen, et al., 2012, Biomaterials 33:6320-6344; and Chen, et al., Stem Cells International, v2015, Article ID 549432; online at: dx.doi.org/10.1155/2015/549432).

Because of complications with whole tooth regeneration, substantial efforts have been made to regenerate the dentine-pulp complex (Gao et al. 2016). However, while adult/postnatal stem cell therapy has been described in many studies, a lack of consensus on the actual efficacy of adult/postnatal stem cells for dentine-pulp regeneration has constrained its clinical value. (Bakhtiar, et al., (2018) Progress in Biomaterials 7:249-268).

Human multipotent mesenchymal stem/stromal cells (MSCs) have been found to modulate allogeneic immune cell responses and induce tolerance, apparently by both the innate pathway (dendritic cell (DC), TNF-a, IL-12, IL-10, and natural killer (NK) cell) and adaptive immunity pathway (T cell, interferon-gamma and IL-4). Specifically, transplanted allogeneic MSCs can be detected in recipients at extended time points, indicating a lack of immune recognition and clearance. Furthermore, a role for bone marrow-derived MSCs in reducing the incidence and severity of graft-versus host disease (GVHD) during allogeneic transplantation has recently been reported. hMSCs were found to alter the cytokine secretion profile of dendritic cells (DCs), naive and effector T cells (T helper 1 [T_{H}1] and T_{H}2), and natural killer (NK) cells to induce a more anti-inflammatory or tolerant phenotype. Specifically, the hMSCs caused mature DCs type 1 (DC1) to decrease tumor necrosis factor α (TNF-α) secretion and mature DC2 to increase interleukin-10 (IL-10) secretion; hMSCs caused T_{H}1 cells to decrease interferon gamma (IFN- [symbol] ) and caused the T_{H}2 cells to increase secretion of IL-4; hMSCs caused an increase in the proportion of regulatory T cells (T_{Regs}) present; and hMSCs decreased secretion of IFN- [symbol] from the NK cells. Mechanistically, the hMSCs produced elevated prostaglandin E2 (PGE₂) in co-cultures, and inhibitors of PGE₂ production mitigated hMSC-mediated immune modulation. (Aggarwal and Pittenger, 2005, Blood. 105(4):1815-1822).

Human umbilical cord-derived mesenchymal stem cells (UC-MSCs) have been reported to be a prospective source for mesenchymal stem cell-based therapy. Reports of MSC-based cell therapy for tissue repair demonstrated that injected MSCs may survive, engraft, and differentiate into specific cell types and repair injured tissues. However, subsequent studies supported the notion that the level of UC-MSCs engraftment in the host organs of recipient animals was low after systemic administration and rather high after local administration. (Arutyunuan, et al., 2016, Stem Cells International, Article ID 6901286; online at: dx.doi.org/10.1155/2016/6901286). This report also proposed mechanisms of UC-MSCs therapeutic activity, including trophic and paracrine effects on cells of the immune system, remodeling of the extracellular matrix, angiogenesis, apoptosis, and stimulation of the migration and proliferation of resident progenitor cells. (Arutyunuan, et al., 2016, Stem Cells International, Article ID 6901286; online at: dx.doi.org/10.1155/2016/6901286).

MSCs have shown promise in research as a potential regenerative therapy, as they have been shown to differentiate into osteoblasts and osteocytes, and they have the ability to recruit hematopoietic host cells when forming bone in vivo. MSCs have been isolated and purified from not only bone marrow, where they cooperate with hematopoietic stem cells (HSCs), but also from a variety of other tissues, such as umbilical cord and umbilical cord blood, white adipose tissue, placenta and the amniotic membrane of the placenta. MSC have also shown potential to integrate into the outer wall of the microvessels and arteries in many organs, such as spleen, liver, kidney, lung, pancreas, and brain. This led to the speculation that both bone marrow- and vascular wall-derived MSC as well as white adipose tissue-, umbilical cord blood-, and amniotic membrane-derived MSC might act as a cell source for regenerative therapy to treat various disorders such as osteoporosis, arthritis, and vessel regeneration after injury. MSC may also be induced to differentiate into functional neurons, corneal epithelial cells, and cardiomyocytes under specific pretreatments ex vivo and in vivo. Some studies have reported that umbilical cord matrix stem cells derived from human umbilical cord Wharton's Jelly were studied for treatments of neurodegenerative disorders such as Parkinson's disease by transplantation into the brain of nonimmune-deficient, hemiparkinsonian rats. Such transplantation resulted in a significant reduction of rotator behavior as a symptom for Parkinson's disease, thus suggesting an additional therapeutic role for umbilical cord matrix stem cells in treating central nervous disorders. These findings were enough evidence for scientists to speculate a promising role for MSC in regenerative therapy. MSC are being used in clinical trials aiming for regeneration of tissues such as bone and cartilage, as well as treatment of disorders such as spinal cord injury, multiple sclerosis (MS), Crohn's disease, and graft-versus-host disease (GvHD) due to their broad differentiation capacity and potential of hematopoietic cell recruitment (Rohban and Pieber, 2017, Stem Cells International, Article ID 5173732; online at: dx.doi.org/10.1155/2017/5173732).

European patent application EP 3446723 and U.S. Pat. No. 9,962,237, naming inventors Nakashima Iohara and Watanabe, describe the use of a dental root canal filling material which includes a serine protease (specifically trypsin) for insertion into the apical side of a root canal subjected to pulp extirpation, or to root canal enlargement and cleaning of an infected root canal. This filling material is also said to be useful for dental tissue regeneration using dental pulp stem cells derived from an individual of middle or advanced age.

Patent Application Publication US 2017/0014454 is directed to systemic, allogenic stem cell therapies for treatment of diseases in animals, in particular, canine patients. treating preselected diseases comprising the steps of providing a therapeutic dose of a mesenchymal stem cell composition, the mesenchymal stem cell composition comprising mesenchymal stem cells harvested from at least one tissue selected from the group consisting of placental tissue, bone marrow, dental tissue, testicle tissue, and dermal tissue; and systemically administering the mesenchymal stem cell composition to the patient suffering from a preselected disease or diseased state through an IV injection. Other than the harvesting of “preselected MSCs” (including dental derived stem cells harvested from dental pulp, periodontal ligaments, and other dental tissues), the publication makes no mention of injection of multipotent stem cells into the oral vasculature at the beginning of the gastrointestinal tract for increasing total systemic health and wellness.

US Patent Application Publication 2008/0133141 entitled “Weighted Scoring Methods and Use thereof in Screening” describes methods for scoring one or more biomarkers in or associated with a test sample and determining a subject's risk of developing a medical condition.

Genetic predisposition and risk can be determined by standard methods of genetic testing. In some embodiments, the subject is homozygous or heterozygous for a certain risk allele. In some such embodiments the methods include genetic testing of the subject for the presence of the risk allele. In other such embodiments the subject has been previously diagnosed for the presence of the risk allele, where such methods may include, without limitation, analyzing a sample of genomic DNA or RNA from the individual for the presence of certain sequences (e.g. an allele on a human chromosome associated risk of coronary artery disease), including SNPs. In some embodiments, the immune or inflammatory disorder is ASCVD.

US Patent Application Publication 2018/0156785, entitled “Biomarkers for determining the clinical response to cell therapy,” describes a method for predicting a clinical response to a therapy based on administration of MSCs in a patient suffering from an immune-mediated inflammatory disease. T cells (CD3/CD4/CD8), NK Cells (CD56+), B cells (CD20+), monocytes (CD14+) and T_{reg} cells (CD4+CD25+FoxP3+) were analysed. Plasma levels of the concentration (in pg/ml) of the cytokines: IL-1-b, sIL-1RA, IL-2, IL4, IL-6, IL-8, IL10, IL12p70, IL17-A, IL23, IFN-α, IFN-γ, sCD-40L, TNF-α, IL-23p19 and TGF-β) were assessed. Patients were then stratified between “responders” and “rest of the population” for treatment with expanded adipose derived stem cells (eASC).

PCT publication WO0223191A1 describes a method comprising assessing the parameters associated with a condition or event associated with the systemic vasculature or assessing a risk of a condition or event occurring, said method comprising contacting a biological sample from a subject to be tested wherein said biological sample comprises one or more members which are present, absent, elevated or otherwise activated in a subject following said condition or event or a condition or event otherwise associated with an aberration wherein said members are selected from two or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, FABP (cardiac fatty acid protein), fatty acid binding protein (FAB protein) including FABP1 and human heart-type, glycogen phosphorylase-BB isoenzyme, .alpha.-atrial natriuretic peptide (ANP), cytoplasmic FABP, brain natriuretic peptide (BNP), adrenomedullin (ADM), low density lipoprotein (LDL), small dense LDL (sdLDL) very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor .alpha. (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, adiponection, Apolipoprotein A (ApoA), Apolipoprotein B (Apo B), apoE, E-selectin, IL-II.alpha., IL-I.beta., IL-4, IL-5, IL-6, IL-8, IL-I.beta., IL-IO, IL-13, IL-18, B-natriuretic peptide (BNP), NT-proBNP, MCP-I, MPO, Intercellular Adhesion Molecule (ICAM), Vascular Cellular Adhesion Molecule (VCAM), soluble vascular cell adhesion molecule-1 (VCAM1), sICAM-1, myeloperoxidase, CD40L, sCD40L, IFN-.gamma., ENA78, fractalkline, PIGF, PAPP-A, RANTES, D-dimer, IMA, FFAu, choline, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, MMP metalloproteinase-9 (MMP-9), metalloproteinase-1 including its tissue inhibitor, angiotensin-converting enzyme, CD95/Apo1/Fas, hepatocyte growth factor, plasma brain natriuretic peptide, angiotensin 11 type receptor, endothelial constitutive nitric oxide synthase, glycoprotein IIIa genetic polymorphisms, factor Vila, vWF, thrombin, endothelin-1, cardiac myofibrillar proteins, Fas and Fas ligand, ligands thereof or binding partners thereof or nucleic acid molecules encoding same or their fragments or ligands or binding partners (or a combination thereof) and contacting said biological sample with a second set of members wherein one or more of said second set of members are binding partners to one or more of said first set of members and wherein the pattern of interaction between said first and second sets of members including the absence of interaction is indicative of said condition or event or a condition or event, and then effecting a suitable treatment regimen.

U.S. Pat. No. 9,002,654 describes a diagnostic method employing multi-analyte analysis of saliva biomarkers as predictors of periodontal and pre-implant disease to determine probability of an oral disease state. The method comprises (a) determining the levels of two or more biomarkers in a sample collected from a first individual, wherein a first biomarker is a bone-specific marker and a second biomarker is a plaque biofilm pathogen marker, said levels of said two or more biomarkers indicating the probability of said oral disease state, wherein the first biomarker is not type I collagen pyridinoline cross-linked telopeptide (ICTP); wherein elevated levels of said two or more biomarkers from said first individual compared to levels of identical biomarkers from a second, healthy individual, or compared to biomarker levels of said first individual measured at an earlier time point are indicative of occurrence of oral disease in said first individual with a probability of diagnosing the disease state equal to or greater than 70%; and (b) treating said oral disease by administering an amount of a therapeutic or prophylactic composition sufficient to reduce activity of said two or more biomarkers. methods of measuring biomarkers to determine the probability of a periodontal and/or pen-implant disease. The biomarkers are selected from the group consisting of Aggregatibacter actinomycetemcomitans, Campylobacter rectus, Fusobacterium nucleatum, Prevotella intermedia, Porphyromonas gingivalis, Tannerella forsythia, Treponema denticola, matrix metalloproteinase-8 (MMP-8), matrix metalloproteinase-9 (MMP-9), osteoprotegerin (OPG), interleukin-1 beta (IL-1.beta.), interleukin-6 (IL-6), interleukin-4 (IL-4), interleukin-10 (IL-10), interleukin-2 (IL-2), interleukin-13 (IL-13), calprotectin, tumor necrosis factor .alpha. (TNF.alpha.) and combinations thereof.

US Patent application publication 20040115701 describes methods of determining the association of a plurality of genes with polypenic disorders and methods of assessing the sensitivity and specificity of the risk of polygenic disorders. In some embodiments, the subject being treated with the disclosed methods is tested for one or genetic markers/alleles that indicate risk of specific disease (e.g., risk of diabetes, CVD, Alzheimer's disease, etc.).

European patent application EP2341131 describes injection of postpartum derived cells (PPDCs) to treat diseases or conditions of bone or cartilage or to augment or replace bone or cartilage. The disease or conditions to be treated include but are not limited to osteoarthritis, osteoporosis, rheumatoid arthritis, chondrosis deformans, dental and oral cavity disease (e.g., tooth fracture and defects), joint replacement, congenital abnormalities, bone fracture, and tumors (benign and malignant). The route of administration can be intravenous (IV). As an alternative to implanting the cells of the invention, or living tissue produced therefrom, the disclosure describes a component or product of PPDCs, such as the extracellular matrix (ECM) or cell lysate produced by those cells for use in tissue repair, replacement or augmentation.

U.S. Pat. No. 10,041,039 describes a method for producing pluripotent stem cell-enriched human dental pulp-derived cells possessing the ability to differentiate into chondrocytes and osteoblasts and the ability to suppress T cell proliferation as well.

US patent application publication 2015/0064141, entitled “Regenerative sera cells and mesenchymal stem cells,” describes three types of skin-derived cells: One type of the cells is mesenchymal stem cells characterized by expression of the cell surface biomarker cluster of differentiation (CD) 146, the second type expresses CD271. The third type of cells is regeneration-associated cells characterized by expression of the cell surface biomarkers stage-specific embryonic antigen 3 (SSEA3) and CD 105 (clone 35). Also disclosed are methods of isolating, purifying, culturing, storing, and using these cells.

US patent application publication 2015/0329827 relates to methods of isolating and expanding pluripotent stem cells, including multi-lineage stress enduring (MUSE) cells (pluripotent, non-tumorigenic stem cells, originally identified in adult human mesenchymal cell populations (Kuroda et all, 2010, Proceedings of the National Academy of Sciences of the United States of America 107: 8639-43)). These cells are stress-tolerant and capable of self-renewing, express a set of genes associated with pluripotency and can be isolated from fibroblasts, bone marrow, or adipose tissues. MUSE cells are attractive sources of autologous cells for regenerative medicine because they do not require genetic manipulation and have low tumorigenic potential (Wakao et al., 2011, Proceedings of the National Academy of Sciences of the United States of America 108: 9875-80.). The pluripotent stem cells of the present invention (e.g., MUSE cells) are capable of differentiating into the three germ layers through in vitro adherent culture. Specifically, the pluripotent stem cells can differentiate into cells representative of the three germ layers, including skin, liver, nerve, muscle, bone, fat, and the like through in vitro induction culture. Also, the pluripotent stem cells are capable of differentiating into cells characteristic of the three germ layers when transplanted in vivo; the pluripotent stem cells are capable of surviving and differentiating into organs (e.g., skin, spinal cord, liver, and muscle) when transplanted to the damaged organs via IV injection into a living body.

However, while these report show the promise of stem cell therapies, none of these studies appear to have noticed any sort of oral-systemic link in disease processes, nor have any reports considered injecting multipotent stem cells into the oral vasculature at the beginning of the gastrointestinal tract for treatment of disease, much less conceived of intravasularly injecting multipotent umbilical stem cells (MUSCs) (without the need for pre-culturing, ex vivo expansion, reprogramming, transformation, immortalization, or inducing the SCs to stimulate and/or activate the cells prior to injection) into the oral vasculature for treating periodontal disease and reducing the inflammatory and/or immune response as the initial step in a system/program for increasing global/multisystem wellness and increasing life quality and life expectancy.

To our knowledge, no one has previously conceived of injection of multipotent stem cells into the oral vasculature at the beginning of the gastrointestinal tract. Furthermore, none of the current research anticipates or even suggests the administration of multipotent stem cells by oral IV injection, a route via which a nearly immediate effect can be observed in the reduction of clinical signs/symptoms of inflammation or infection, reduction of bacterial load during infection, reduction of pain, treating periodontal disease, reduction of bone loss and/or stimulating new bone growth, reduction of dental pocket depth, reduction of point bleeding, as well as reduction of many other objective symptoms of disease or other improvement in health and multisystem wellness (e.g., tissue regeneration). This method of IV injection of multipotent stem cells and the associated system/protocol can test for and/or identify a variety of diagnostic indicators, including: pathogenic bacteria (e.g., Porphyromonas gingivalis (P.g.)), viruses and other infectious agents that cause periodontal and odontogenic inflammation and/or infection, as well as assaying for the presence or absence of any one or more host-specific biomarkers (e.g., genes or genetic mutations, proteins, and/or small functional RNAs). In some instances, a sample of a bodily fluid (e.g., blood, plasma, serum, gingival fluid, sputum, saliva, wound exudate) can be drawn from and/or to determine baseline levels before and after treatment. In some instances, post-treatment engraftment of the injected multipotent stem cells is assessed.

The present disclosure is directed to the therapeutic use of multipotent stem cells (e.g., multipotent umbilical stem cells (MUSCs)), administered intravenously into the vasculature of the oral cavity, according to the system and methods/protocol provided herein. The success of these methods and the treatment protocol provided is monitored using specific laboratory-based metrics. The therapeutic use of such stem cells that are multipotent, without the need for reprogramming or inducing, is demonstrated herein to be effective in improving the symptoms and outcomes of not only oral and dental diseases but also systemic diseases. With the presently described assessment-driven therapy, significant improvements in a patient's oral/periodontal health, as well as increases in overall systemic and multisystem wellness are achieved.

To evaluate the therapeutic effect of in administration of multipotent stem cells, a patient's blood pressure can be measured, and a sample of the patient's blood may be drawn and complete blood count (CBC), metabolic panel, and lipid profile, and cholesterol, LDL and HDL levels assessed post-injection.

Disclosed herein are methods of using multipotent stem cells, e.g., MUSCs, in a system/protocol for treating disease, improving health and enhancing global/multisystem wellness. Treatment begins within the digestive tract as an intravenous (IV) injection within oral cavity. The oral cavity is extremely vascular, making it a prime candidate for simple intravascular injection at the site of initial concern (in the case of periodontal disease). Additional advantages are the rapid (nearly immediate) uptake of the stem cells into the vasculature supplying the first part of the gastrointestinal tract for quick systemic absorption. In the dental office, this procedure is no more invasive than receiving a traditional numbing agent before dental work, and therefore the patient has very little down time. Another advantage of the system described herein is that the multipotent stem cells (e.g., multipotent umbilical stem cells (MUSCs) available from Invitrx Therapeutics, Lake Forest, Calif.) are already activated without the need for pre-culturing, reprogramming, stimulating, activating, or inducing the multipotency of the cells in vitro before injection. Using commercially available multipotent stem cells (of good quality with a high concentration of living stem cells) obviates the need for cumbersome steps of treating and activating MSCs or autologous cells before oral IV-injection.

The present disclosure describes the discovery that, upon administration of MUSCs by oral IV injection, a nearly immediate effect can be observed in the reduction of clinical signs/symptoms of inflammation or infection, reduction of bacterial load during infection, reduction of pain, treating periodontal disease, reduction of bone loss and/or stimulating new bone growth, reduction of dental pocket depth, reduction of point bleeding, as well as reduction of many other objective symptoms of disease or other improvement in health and global multisystem wellness (e.g., tissue regeneration). This method of IV injection of MUSCs and the associated system/protocol can test for and/or identify a variety of diagnostic indicators, including: pathogenic bacteria (e.g., Porphyromonas gingivalis (P.g.)), viruses and other infectious agents that cause periodontal and odontogenic inflammation and/or infection, as well as assaying for the presence or absence of any one or more host-specific biomarkers (e.g., genes or genetic mutations, proteins, and/or small functional RNAs). In some instances, a sample of a bodily fluid (e.g., blood, plasma, serum, gingival fluid, sputum, saliva, wound exudate) can be drawn from the patient to measure baseline levels before treatment and/or test for post-treatment improvements (e.g., engraftment of the injected MUSCs, and/or improvement in one or more of the biomarkers and/or indicators of disease).

Autoimmune diseases can also be detected orally. White autoimmune Purple Heart disease describes a specific appearance of gum tissue assess clinically in a dental office, in which specific differences of color (white, blue/purple) and texture are subtle signs in the mouth of disease. Generalized red-pink color may indicate systemic inflammation and/or acid influx from the stomach/GI tract. Such hues in the anterior region of the mouth, as well as other symptoms (including web-like and/or peeling of outer layer of connective tissue) signal the dental practitioner to consider not only heart disease but also autoimmune disease.

### Definitions

As used throughout the present disclosure, the technical and scientific terms used in the descriptions herein will have the meanings commonly understood by one of ordinary skill in the art, unless specifically defined otherwise. Accordingly, the following terms are intended to have the following meanings:

As used in this specification and the appended claims, the singular forms “a,” “an,” and “the” include plural referents unless the context clearly indicates otherwise. Thus, for example, reference to a subject's “gene” can include more than one gene, reference to a “device” includes a single device as well as two or more of the same or different devices, and reference to a “test” refers to a single test as well as two or more tests. The use of “or” should be understood to mean “and/or” unless stated otherwise. Similarly, “comprise,” “comprises,” “comprising” “include,” “includes,” “including,” “has,” “have” and “having” are interchangeable and not intended to be limiting. It is also to be understood that where descriptions of various embodiments use the term “comprising,” those skilled in the art would understand that in some specific instances, an embodiment can be alternatively described using language “consisting essentially of” or “consisting of.” For example, any multipotent or pluripotent cells (already activated/immortalized) may be used. Furthermore, as used herein, the phrase “consisting essentially of” means that a composition may include other non-active, non-essential components in addition to active/essential elements or components. Moreover, due to biological functional equivalency considerations, changes can be made in non-essential components of the compositions without affecting the biological action, in kind or amount, as described herein. All such modifications are intended to be included within the scope of the appended claims.

As used herein, “treatment”, “treating”, “treat” and the like are used herein to generally refer to obtaining a desired pharmacologic and/or physiologic effect, such as the patient's relief from pain and/or alleviation of pathological processes. In the context of the present disclosure as it relates to any of the conditions, diseases, disorders, infections and/or inflammatory responses recited herein, the terms “treat,” “treatment,” and the like mean to relieve or alleviate one or more symptoms associated with such condition, or to slow or reverse the progression or anticipated progression of such condition, such as slowing the progression of a disease or disorder, an inflammatory response and/or increasing the clearance of an infectious organism, so as to alleviate/reduce the symptoms caused by the disease, disorder, inflammation or infection (e.g., reduction in bleeding point in the oral cavity, pocket depth, pathogen burden, etc.). Thus, the effect can be prophylactic in terms of completely or partially preventing a disease or symptom thereof, or may be therapeutic in terms of a partial or complete stabilization or cure for a disease, one or more symptoms and/or adverse acute or chronic effect(s) attributable to the disease (e.g. coronaviral infections such as COVID-19). Such treatment covers any improvement in a manifestation of the disease in a mammal, particularly a human, and includes: (a) preventing the disease or symptom from occurring in a subject which may be predisposed to the disease or symptom but has not yet been diagnosed as having it (e.g., genetically, history of related disease or disorder, etc.); (b) inhibiting the disease and/or symptoms from further progressing (e.g., arresting the disease at its current stage of development/progression); or (c) relieving one or more disease symptoms, i.e., causing regression of the disease or symptom. Those in need of treatment include individuals already diagnosed with, for example, diabetes, as well as those in which the disease is to be prevented, such as an individual having pre-diabetes but not yet diagnosed with diabetes per se.

“Ameliorating” or “ameliorate” refers to any indicia of success in the treatment of a pathology or condition, including any objective or subjective parameter such as abatement, remission or diminishing of symptoms or an improvement in a subject's physical or mental well-being. Amelioration of symptoms can be based on objective or subjective parameters; including the results of a physical examination and/or a psychiatric evaluation.

Efficacy of treatment or amelioration of disease can be assessed, for example by measuring disease progression, disease remission, symptom severity, reduction in pain, quality of life, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration MUSCs (or a composition comprising the MUSCs) of the present disclosure, “effective against” a disease or disorder is meant to indicate that administration in a clinically appropriate manner results in a beneficial effect for at least some percent or fraction of patients receiving the treatment, such as an improvement of symptoms, a cure, a reduction in disease/pathogen load, reduction in inflammatory cell numbers, an extension of life, an improvement in comfort and/or quality of life, a reduction in pain, a reduction in the need for other medications or more invasive treatment, or other beneficial effect generally recognized as positive by medical doctors familiar with treating the particular type of disease, disorder, inflammation or infection.

A treatment or preventive effect is evident when there is a statistically significant improvement in one or more parameters of disease status, or by a failure to worsen or to develop symptoms where they would otherwise be anticipated. As an example, a favorable change/improvement of at least 10% in a measurable parameter of disease, and preferably at least 20%, 25%, 30%, 40%, 50%, 60%, 75%, 85%, 95% or more can be indicative of effective treatment. Efficacy for the MUSCs or formulation of MUSCs can also be judged using an experimental animal model for the given disease as known in the art. When using an experimental animal model, efficacy of treatment is evidenced when a statistically significant modulation in a marker or symptom is observed.

By “reduce,” “lower,” “inhibit,” or “decrease” in the context of a disease marker, a parameter or a symptom of a disease, disorder, inflammation and/or infection is meant a statistically significant decrease in the level or activity of the marker, parameter or symptom. Preferably, the reduction is from a level generally understood to be indicative of a diseased, inflamed and/or infected state down to a level accepted to be within the range of normal for an individual without such disease, disorder, inflammation and/or infection, or as compared to the patient's baseline measurement. The decrease varies depending on the clinical observation and/or parameter being measured, and can be, for example, at least 10%, at least 20%, at least 30%, at least 40% or more, at least 50% or more, at least 60% or more, at least 70% or more, at least 80% or more, at least 90% or more, at least 92% or more, at least 95% or more, or a 100% lowering or reduction of the marker or symptom.

By “increase,” “elevate” or “raise” (in the context of alleviating a disease, or a symptom associated with a disease), is meant a statistically significant increase in a level of a biomarker or improvement a symptom, which increase or improvement is generally understood to be indicative of an improved state of health and wellness. The increase is preferably up to a level accepted as within the range of normal for an individual without such disease, disorder, inflammation and/or infection, or as compared to the patient's baseline measurement, and can be, for example, at least 10%, at least 20%, at least 30%, at least 40% or more, at least 50% or more, at least 60% or more, at least 70% or more, at least 80% or more, at least 90% or more, at least 92% or more, at least 95% or more, a 100% or more, at least 150% or more, at least 200% or more, or a 400% increase or rise in level of the marker or improvement of a symptom associated with an improved state of health and wellness.

For example, but without limitation, a measurement and/or score for a given parameter can be designated “high” or “low” (meaning above or below, respectively) as compared to (i) a standard/reference score, (i) a normalized average score derived empirically from a group of healthy or unhealthy individuals, (iii) a score with an arbitrary unit assigned to correlate with a treated or untreated condition of a subject, or (iv) an ideal and/or desired score.

As used herein, the terms “chart,” “matrix,” “objective metric,” “clinical calculator” and “nomogram” refer to the tools exemplified in Table 1 and FIG. 1, which are used to quantify and assign a weighted score with regard to a patient's health and wellness or disease status. Weighted scores can be based on a numeric scale (e.g., 1 to 10, 1 to 100, or 1 to 1000), or on percentages, percentiles within a population of subjects, actual scores in standard usage in the clinic or laboratory to measure a given parameter, or even heat maps with color ranges and coding where cool colors (e.g., blues, greens, dark purples; or lighter greys in a monochromatic greyscale) represent low values, mid-range/medium colors (e.g., oranges, pinks and tinted colors or medium greys in a monochromatic greyscale) represent moderate or average values on a scale, and warm colors (e.g., reds, yellows, whites and highly tinted colors or darkest greys using a monochromatic greyscale) represent high values. On the charts presented herein, each parameter can be assessed and assigned a weighted score, which can then be used in a calculation (e.g., addition or multiplication of the values for each box or column in the chart, and/or subjected to a formula/algorithm) to generate a multisystem score. In some instances, an equation, algorithm or formula is used to assess and assign a score to a patient. In some embodiments, the nomogram allows quantification of severity of APS. For example, some formulas for measuring biomarkers can be found in US Patent Application Publication 2008/0133141, incorporated by reference in its entirety.

As used herein, the phrase “therapeutically effective amount” refers to an amount that provides a therapeutic benefit in the treatment, prevention, alleviation, improvement in, or management of a pathological process and/or a symptom associated with a disease, disorder, inflammation and/or infection. The specific amount that is therapeutically effective can be readily determined by an ordinary medical practitioner and may vary depending on such as, for example, the type of pathological process, the stage of the pathological process, or the patient's dental and/or medical history, age, weight or state of health and wellness as indicated by measurable laboratory assessments/tests as described herein. The specific amount that is therapeutically effective also can be readily determined based on the administration of other agents that have been used or are currently being used to treat the pathological process, disease, disorder, inflammation and/or infection.

The terms “recipient,” “individual,” “subject,” “host” and “patient” are used interchangeably herein and refer to any mammalian subject for whom diagnosis, treatment, or therapy is desired, particularly humans. “Mammal” for purposes of treatment refers to any animal classified as a mammal, including humans, primates, rodents, domestic and farm animals, and zoo, sports, or pet animals, such as dogs, horses, cats, cows, sheep, goats, pigs, etc. In most embodiments, the mammal is human.

The age of the subject may vary, especially depending on the type of mammal being treated. Where the mammal is a human, the age of maturity deemed to be “adult” is often 18 years or older. In some instances, the age of an adult human is 21 years old or older. In some instances, the patient is an adult. In some instances, the patient is an individual suffering from periodontitis. In some instances, the patient has early stage gum disease and is at risk of developing periodontitis. In some instances, the patient may be one in whom it has been determined, e.g., in the form of receiving a diagnosis, that the patient is suffering from or at a higher than average risk of developing one or more of cardiovascular disease, diabetes or pre-diabetes, migraine, cognitive impairment, Alzheimer's disease, dental/periodontal diseases, dental caries and/or tooth decay. At risk individuals may be less than 20 years of age (y.o.a.), between 20 and 29 (y.o.a.), between 30 and 39 (y.o.a.), between 40 and 49 (y.o.a.), between 50 and 59 (y.o.a.), between 60 and 69 (y.o.a.), between 70 and 79 (y.o.a.), between 80 and 89 (y.o.a.), between 90 and 99 (y.o.a.), or over 100 (y.o.a.). A patient may be e.g., about 50 years old or older, about 60 years old or older, about 70 years old or older, about 80 years old or older, about 90 years old or older, and sometimes about 100 years old or older. In some cases, the patient is between the ages of about 50 and 100, or between 60 and 90, or between 75 and 85 years of age.

Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, the condition, disorder or disease state being assessed and/or the parameter being measured. In other words, cells present in adult mammals treated in accordance with methods of the invention will become more responsive to cues, e.g., activity cues, which promote improved multisystem health and wellness.

An “effective amount” is an amount sufficient to effect beneficial or desired clinical results. An effective amount can be administered in one or more administrations or doses. For purposes of the present disclosure, an effective amount of a therapeutic agent is an amount that is sufficient to palliate, ameliorate, stabilize, reverse, prevent, slow or delay the progression of the immune and/or inflammatory disease state, e.g. atherosclerosis or atherosclerotic plaque. For example, the percent of aortic surface area with atherosclerotic plaque may be reduced 25%, 50%, 75% or more relative to a control individual. Similar effects and comparable measurements indicating a reduction in the disease state may be obtained using such indicia appropriate for human patients, including without limitation C-reactive protein (CRP) and fibrinogen; lipoprotein-associated phospholipase A2 (Lp-PLA2) and myeloperoxidase (MPO); growth differentiation factor-15 (GDF-15) inflammatory markers; ambulatory arterial stiffness, IVUS imaging, and the like. See, for example Krintus et al. (2013) Crit Rev Clin Lab Sci. 11:1-17; Kollias et al. (2012) Atherosclerosis 224(2):291-301; and Kollias et al. (2011) Int. J. Cardiovasc. Imaging 27(2):225-37, each herein incorporated by reference.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations. The definition also includes sample that have been enriched for particular types of molecules, e.g., nucleic acids, polypeptides, etc.

The multipotent stem cells can be administered as the sole therapeutic agent, or in combination with one or more additional therapeutic agents as a part of the same or a separate composition, and/or by another method described herein.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, the multipotent stem cell composition is are administered and after a period of time, and then a new assessment of the biomarkers and wellness status is performed. In some embodiments, the period of time is 1-5 minutes, 10 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks. Other therapeutic agents may be concomitantly, or subsequently administered in addition to the stem cells.

The present disclosure addresses a long-felt need for a system and method involving testing, treating, and achieving results in preventing cardiovascular diseases alongside periodontal disease; it cleverly unites state-of-the-art approaches in both dentistry and medicine via a unique simultaneous assessment of cardiovascular, inflammatory and dental indicators. The system and method of global, multisystem wellness assessment herein disclosed incorporates both dental and medical histories, a subject's family history and genetics, careful observations of clinical signs, patient self-reporting and periodontal and OralDNA testing for pathogens, oralDNA testing of genetic markers of risk factors, results from test strips for testing pH, for example, (as well as other parameters) in saliva or urine, medical blood work or advanced lipid testing, Carotid Intima-Media Thickness (CIMT) ultrasound, inflammatory markers, and other genetic tests, and clinical exam of physician/cardiologist, which data-gathering results in a global, multisystem landscape for an oral-systemic healthcare provider and patient to find suggestions/prescriptions and guidance toward a healthier overall state of wellness.

In the method, the dentist/physician reviews the pre-injection data, reports and discusses any and all appropriate therapeutic measures with the patient, takes immediate action with regard to urgent needs (including oral intravenous administration of multipotent stem cells) where appropriate (e.g., in cases of extreme inflammation and/or pain in the oral cavity) and develops an ITP and prescription for repeated treatments as well as other dental and medical interventions as necessary, plans and/or performs post-injection test(s), including imaging studies and/or other diagnostic tests. The results and data interpretation (pre- and post-injection) have a material impact on the well-being of the patient as well as such patient's life expectancy. Given the nature and the complexity of the specialized tests performed with respect to the patient, the referring physician may seek consultations.

In some embodiments, this method of treatment and system enhance oral and multisystem health and wellness as measured by radiographic bone regeneration, decreased levels of pathogens, decrease of inflammatory markers observed via blood work, decreased zone of inflammation via thermography, carotid CIMT. The results can be observed over as short a period of minutes, hours, or days, or can be observed over longer periods such as weeks, months or years.

An oral-systemic link is well established, but, to date, no system has been developed to assess medical and dental aspects of chronic inflammation together, to develop a multisystem intervention/treatment/prevention plan. The presently disclosed comprehensive approach to wellness does not divide the body into part and pieces, but remains cognizant that the body functions as a whole. Chronic inflammation stimulates free radicals and oxidative stress catalyze metabolic diseases that lead to CVD, diabetes, metabolic syndrome, insulin resistance, cancer, premature aging, autoimmune diseases, strokes, periodontal disease, etc. Even natural aging and imbalances in hormones, stress, chronic inflammation, acid reflux, sleep disorders, nutritional deficiencies, early signs of diabetes, and cancers can be seen in the oral cavity.

As the start of the digestive system, the oral cavity plays an important role in wellness; oral environment is affected by pH from prescriptions, dry mouth, diabetes, food ingested, physical oral care, and is one of the only direct accesses of bacteria into the blood stream via the vascular area directly around the teeth. While a few other entry zones exist, such as open wounds and the vasculature of digestive/gastrointestinal tract, the importance of the oral-systemic link is a crucial consideration to overall health and wellness, and has remained underdiagnosed. Nonetheless, it is clear that the burden on the body from early through late stage periodontitis, and/or undiagnosed dental abscesses prevent systemic health from ever being optimal.

This simultaneous, complimentary (and potentially synergistic) approach of medical and dental assessment of wellness in multiple systems of the body and development of targeted interventions to decrease inflammation can achieve far better results for a subject's global/multisystem health and wellness than has been achieved with medicine and dentistry as separate practices.

Continued re-testing after certain time intervals allows all participants to observe and understand the subject's individual response to the prescribed intervention/treatment/maintenance therapy and preventive care. Interventions can include nutrition, changes from a sedentary to a more active lifestyle, weight loss, quitting smoking, quitting drinking alcohol, and even replacing silver-mercury fillings can all have an impact on one's multisystem health and wellness; these can benefit most individuals, but using the systems and methods disclosed herein to design particular programs of intervention targeted to particular states of illness or predisposition to illness, one can arrive at even better, more ideal states of health and wellness.

As used herein, cardiovascular disease is associated with or resulting from atherosclerosis, and can involve coronary artery disease (CAD), ischemic heart disease (IHD), angina pectoris, AMI, death, stroke or peripheral vascular disease. The phrase “coronary artery disease” refers to atherosclerotic disease of the coronary arteries, but also infers probable atherosclerotic disease of the peripheral arteries such as those supplying the legs and the brain, and includes the consequences of CAD, such as myocardial infarction and death, angina pectoris, stroke and peripheral vascular disease.

Lung diseases such as asthma, acute lung disease, chronic obstructive pulmonary disease (COPD) and respiratory infections (e.g. due to coronaviral infection, for example SARS, MERS or COVID-19) are also treatable according to the methods described herein.

The term “biomarker” refers to a distinctive biological or biologically derived indicator of a process, event, or condition. Biomarkers as used herein encompass, without limitation, gene products, including proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures that are associated with a biological state. Biomarkers can also include mutated proteins or mutated nucleic acids. Biomarkers preferably include inflammatory, infection, thrombotic or autoimmune biomarkers. Many biomarkers can be observed using OralDNA or Ayass testing.

The phrase “inflammation biomarker” or “biomarker of inflammation,” as used herein, refers to a biomarker that is an indicator of inflammation. Exemplary inflammation biomarkers include C-reactive protein (CRP), interleukin 1 composite genotype, interleukin (IL)-6, interleukin 17A, beta-defensin 1, CD14, tumor necrosis factor alpha, toll-like receptor 4 composite genotype, matrix metalloproteinase 3, and serum amyloid A protein, homocysteine.

The phrase “biomarker of infection” or “infection biomarker,” as used herein, refers to a biomarker that is an indicator of infectious diseases. A list of potential infection biomarkers (biomarkers of infection) can be found at the Infectious Disease Biomarker Database (IDBD) (See biomarker.cdc.go.kr and biomarker.korea.ac.kr). Exemplary infection biomarkers include gene products of (including proteins): CRP, anti-cytomegalovirus (CMV) antibody, Chlamydia pneumonia, herpes simplex virus (HSV) types 1 and 2, Helicobacter pylori, and hepatitis A virus, as well as periodontal pathogens.

“Autoimmune disease biomarker” or “biomarker of autoimmune disease,” as used herein, refers to a biomarker that is an indicator of autoimmune disease. Three groups of gene products, e.g., proteins, are reflective of the autoimmune disease process: (1) degradation products arising from destruction of affected tissues, (2) enzymes that play a role in tissue degradation and (3) cytokines and other proteins associated with immune activation (Prince, H. E., Biomarkers, 2005, November 10, Suppl 1: S44-49). Exemplary autoimmune disease biomarkers include gene products of (including proteins): antibody to Heat Shock Protein 60 (anti-HSP60), Heat Shock Protein 70 (HSP70), aggrecan fragments, C-propeptide of type II collagen and cartilage oligomeric matrix protein, matrix metalloprotease (MMP)-I, MMP-3 and MMP-1/inhibitor complexes thioredoxin, IL-16 and tumour necrosis factor (TNF)-alpha, neurofilament light protein and glial fibrillary acidic protein, MMP-2 and MMP-9 and TNF-alpha and soluble vascular adhesion molecule-1.

“Cellular stress biomarker,” or “biomarker of cellular stress,” as used herein, refers to a biomarker, the levels of which increase when a cell is exposed to stress. Exemplary cellular stress biomarkers include Heat Shock Protein (HSP) 70 (HSP70), and certain other HSPs, such as HSP32, HSP27, HSP72, HSP90, HSP47, as well as ubiquitin, and Hsc70, and cellular stress biomarkers discussed by Rajdev and Sharp, Toxicologic Pathology, 28(1) 105-112 (2000); and Zhou et al, Circulation, 110: 207-213 (2004).

The phrase “biomarker of thrombosis,” or “thrombosis biomarker,” as used herein, refers to a biomarker that is an indicator of thrombosis. Some examples include fibrinogen, prothrombin 1.2, tissue plasminogen activator antigen (tPA), plasminogen activator inhibitor-1 (PAI-1), and FDP markers, such as an FDP marker that includes a mixture of at least two fibrin and fibrinogen degredation products (FDPs), such as two or more of fragments X, Y, D, D-dimer, and E fragment Y, and initial plasmin digest products (IPDP).

As used herein, the phrases/terms “fibrin degradation product(s),” “fibrin and fibrinogen degradation product(s),” and “FDP” refer to one or more fragments produced when either fibrin or fibrinogen is degraded. FDPs include four principal FDPs, called X, Y, D, and E fragments (fragment X, fragment Y, fragment D, and fragment E) that are liberated in various combinations. Cleavage of fibrinogen by plasmin produces fragments D and E as the primary end-products. Thrombin converts fibrinogen to fibrin. When a fibrin clot is broken down by plasmin, the last fragment to be degraded (containing two D and one E subunits) is split, releasing the E fragment and also two D fragments covalently linked together (called “D-dimer”). This D-dimer is produced from fibrin, not fibrinogen degradation. Accordingly, the FDP biomarker can include the presence of one or more of X, D, and E fragments. In one example, the FDP biomarker includes fragment Y; in one example, it includes one or two distinguishable forms of initial plasmin digest product (IPDP). In some embodiment, the provided methods and systems detect a the level of a FDP marker; in one aspect, this FDP marker includes a mixture of at least two FDPs, such as fragments D, E, and D-dimer, and in some aspects further including one or more of fragments X, Y, and IPDP. In one example, the FDP marker includes one, more, or all FDPs detected by the DR-70 ELISA assay. In another example, the FDPs include one or more FDPs described in International Application Publication Number WO 2010/114514 A1. In another example they include one or more FDPs detected by a Fibrinogen ELISA assay using either anti-Fibrinogen polyclonal or multiple monoclonal antibodies.

“C-reactive protein” is also known as “hsCRP”, or “CRP,” and is a marker of the reactant plasma protein component of the inflammatory response. CRP is a protein produced by hepatocytes as part of the non-specific acute phase response to inflammatory conditions. It is used to diagnose and monitor a wide variety of infectious diseases.

“Heat shock protein 70” is also known as“HSP70” “HSP73” “HSPA8” Other family members include HSP 70-1, HSP 70-2, HSP 70-4, HSP 70-4L, HSP 70-5, HSP 70-6, HSP 70-7, HSP 70-8, HSP 70-9, HSP 70-12a, HSP 70-14. Increased levels are found during conditions in which cells are exposed to stress. Thus, HSP70 is among the cellular stress biomarkers.

“Cytomegalovirus” “CMV” is a part of the herpes family of viruses. Other family members include herpes simplex virus type 1 (HSV-1 or HHV-1) and herpes simplex virus type 2 (HSV-2 or HHV-2), varicella zoster virus (VZV), human herpesvirus (HHV)-6, HHV-7, and HHV-8. The biomarker detected for CMV includes CMV antibody (CMV-Ab).

Exemplary pathogens associated with disease and identified in OralDNA testing include, but are not limited to: Aggregatibacter actinomycetemcomitans, Campylobacter rectus, Capnocytophaga species (gingivalis, ochracea, sputigena), Eikenella corrodens, Eubacterium nodatum, Fusobacterium nucleatum/periodonticum, Peptostreptococcus (Micromonas) micros, Porphyromonas gingivalis, Prevotella intermedia, Tannerella forsythia, Treponema denticola, and combinations thereof. Results from assays monitoring these pathogens can be used in the presently described method and system in order to diagnose, develop an ITP, or predict the risk/probability of occurrence of a disease in a subject.

The term “mammal” or “patient” or “subject” refers to such organisms as mice, rats, rabbits, goats, horse, sheep, cattle, cats, dogs, pigs, preferably domesticated animals such as pets, more preferably monkeys and apes, and most preferably humans. In some embodiments, the subject is a human, and the test or biological sample, which can be a test sample or a control sample, used is a bodily fluid or bodily tissue.

The terms “bodily fluids” or “body fluids” as used herein include circulating and non-circulating fluids. Examples of circulating fluids include blood, serum, CSF, and lymph fluid. Examples of non-circulating fluids include synovial fluid. Body fluids can include amniotic fluid, aqueous humour, vitreous humour, breast milk, cerebrospinal fluid (CSF), cerumen (earwax), chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, serum, sputum, sweat, synovial fluid, tears, vomit, urine or exhaled condensate.

As used herein, a “digital wellness landscape” refers to digitized information about the medical and dental data points representing all that is known about a subject. A digital wellness landscape is obtained, for example, using a computer and appropriate software, data entry from medical and dental histories, oral sampling and imaging as well as whole-body imaging techniques, including but not limited to magnetic resonance imaging, laser scanning, ultrasound, x-ray, etc. The digital wellness landscape described herein can be stored in computer-readable form. The digital wellness landscape is generally capable of being represented visually and/or graphically on a computer screen or video monitor.

“Display” refers to a computer screen, video monitor, or other device capable of presenting an image to a viewer. “Display is capable of being manipulated” means that the image can be adjusted, elements added or moved on the screen or monitor to simulate, predict the effects of, or prescribe various adjustments to image.

The term “computer-readable medium” as used herein refers to any medium that participates in providing instructions to a processor for execution. Such a medium may take many forms, including but not limited to non-volatile media, volatile media, and transmission media. Non-volatile media may include, for example, optical or magnetic disks. Volatile media may include dynamic memory. Transmission media may include coaxial cables, copper wire and fiber optics. Transmission media may also take the form of acoustic, optical, or electromagnetic waves, such as those generated during radio frequency (RF, e.g. using an RFID tag) and infrared (IR) data communications. Common forms of computer-readable media include, for example, diskette, hard disk, magnetic tape, or other magnetic medium, CD-ROM, CDRW, DVD, or other optical medium, RAM, PROM, and EPROM, FLASH-EPROM, or other memory chip or cartridge, a carrier wave, or other medium from which a computer can read.

“Electronically transmitting the digital wellness landscape” refers to the act of conveying the digitized anatomical information as electromagnetic radiation through an through wires, coaxial cables, dielectric slabs, optical fibers, electric power lines, and waveguides or wireless media to a receiver or storage device, which may reside at a site remote from that at which the digital wellness landscape originates. Similarly, digitized wellness information may be sent from a receiver or storage device to a site at which a digital wellness landscape can be obtained, and/or to a site at which a maintenance plan/treatment course can be prescribed by a clinician/physician/dentist.

“Data compression” refers to the process of encoding information using fewer bits (or other information-bearing units) than an unencoded representation would use through use of specific encoding schemes.

“Securing” or “security encoding” refers to the process of encrypting information for protection of the subject's personal medical and dental history and digital wellness landscape information.

“Initial digital wellness landscape” refers to the sum of all the data points in a first, untreated or restored state before the analysis and design of a subject's wellness treatment plan in accord with the present methods and systems. “Restored digital wellness landscape” refers to the treated or restored state that is a physiologically or medically desired, improved state of multisystem wellness achieved during or subsequent to completion of the treatment plan designed in accord with the present methods and systems. “Intermediate wellness state” or “intermediate state” refers to the subject's state after treatment designed in accord with the methods and systems described herein, where the treatment adjusts one or more medical or dental health parameters to achieve a wellness status that is different from the initial digital wellness landscape status, yet is not necessarily at the desired, ideal restored state of global/multisystem wellness. An image of a subject can be obtained using a means of imaging selected from, for example, magnetic resonance imaging (MRI), computed tomography (CT), radiologic imaging such as x-rays, ultrasound imaging, infrared imaging, or any variations or combinations thereof.

“Superimposition of the digital wellness landscapes” refers to placement of an image or video representing a second digital wellness landscape on or over a first image or video representing a digital wellness landscape for comparison of two or more digital wellness landscapes. In some embodiments, the superimposition of the digital wellness landscape images aligns one or more data points in each image. As can be appreciated, superimposition of two images permits assessment of differences between an initial and an intermediate or a restored wellness state, and informs the measurements and/or calculations for design of multisystem wellness plan/map toward maintenance or treatment.

“Simulation of a movement path” or “defining one or more movements of any data point to move from an initial state to the desired restored state” refers to the process of measuring or calculating the direction/course/path of actions (i.e., a series of behavioral steps) needed for the subject to get from an initial or intermediate wellness state at a given time point to an intermediate or restored state at a later time. The movements can be a distance in one or more of the X, Y, Z directions, or can be angular movements around the X, Y, Z axes.

A “signal” can refer to an electronic event, message, or data packet that can carry varying quantities of information and which is transmitted between computational processes. As used herein, a signal can be sent from a computer running an associated software program, to another computer, which can process the signal and which can then send a signal to one or more receiving parties, e.g., patient, doctor or dentist.

“Timer for scheduling a subsequent obtaining of an intermediate wellness state at a given time point to an intermediate or restored state at a later time or follow-up appointment” refers to a step of the method in which a desired length of time of treatment with the wellness program/plan has passed and the subject's wellness state is re-assessed for design of the next step in treatment. For example, the timer can prompt the user (patient/clinician/physician/dentist) to schedule an appointment with the treating clinician, physician or dentist to reassess and monitor progress or to design a new treatment plan.

“Treatment plan” and ITP refer to a design of one or more courses of dental treatments, follow-up assays, medications, dental or medical surgeries, and/or assigned/prescribed changes in behavior to achieve a desired global systemic state of wellness.

“Labeled in order of use” refers to markings on the output treatment plan that indicate the sequential order in which the prescribed treatment steps are to be performed.

“Optional” or “optionally” refers to a subsequently described circumstance that may or may not occur, so that the scope of the embodiment includes instances where the circumstance occurs and instances where it does not.

Having a “predisposition to develop a disease or disorder” means that a subject having a particular genotype and/or haplotype has a higher likelihood than one not having such a genotype and/or haplotype for developing a particular disease or disorder.

“Associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

When a range of values is provided, it is understood that each intervening value, to the tenth of the unit of the lower limit unless the context clearly dictates otherwise, between the upper and lower limits of that range is also specifically disclosed. Each smaller range between any stated or intervening value in a stated range and any other stated or intervening value in that stated range is encompassed by the disclosure. The upper and lower limits of the smaller ranges can be independently included or excluded in the range, and each range where either, neither or both limits are included in the smaller ranges is also encompassed by the disclosure, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

The patents, patent applications and non-patent publications discussed herein (each of which is incorporated by reference in its entirety) are provided for their disclosure as background information, including contemporaneously known methods and/or materials without any admission of the publication's status as prior art. Nothing herein is to be construed as an admission that the present invention is not entitled to antedate such publication; furthermore, the publication dates provided may be different from the actual dates of publication, which may require independent confirmation. It is understood that the present disclosure supersedes any disclosure of an incorporated publication to the extent there is a contradiction.

Inflammatory Indications: In some embodiments, the methods and compositions of the present disclosure may be used to treat subjects with diseases, disorders and/or conditions related to inflammation. Inflammation may be upregulated during the proteolytic cascade of the complement system. Although inflammation may have beneficial effects, excess inflammation may lead to a variety of pathologies. Accordingly, the MUSC compositions of the present disclosure may be used to reduce or eliminate inflammation associated with complement activation.

Sterile Inflammation: In some embodiments, the methods and compositions of the present disclosure may be used to treat, prevent or delay development of sterile inflammation. Sterile inflammation is an excessive inflammatory response “gone awry” that occurs when there is no threat from invading microorganisms/infection, but rather occurs in response to stimuli or stressors such as genomic stress, hypoxic stress, nutrient stress or endoplasmic reticulum stress caused by a physical, chemical, or metabolic noxious stimuli. Sterile inflammation may contribute to pathogenesis of many diseases such as, but not limited to, tissue destruction seen in myocardial infarction, stroke, ischemia-induced injuries, rheumatoid arthritis, acute lung injuries, drug-induced liver injuries, inflammatory bowel diseases and/or other diseases, disorders or conditions. (Prockup, 2013, Stem Cells, 31:2042-2046). The mechanism of sterile inflammation, as well as additional methods and compositions for treatment, prevention and/or delaying of symptoms of sterile inflammation may include any of those taught by Rubartelli et al. in Frontiers in Immunology, 2013, 4:398-99; Rock et al. in Annu Rev Immunol. 2010, 28:321-342; or in U.S. Pat. No. 8,101,586, the contents of each of which are herein incorporated by reference in their entirety.

Systemic Inflammatory Response (SIRS) and Sepsis: In some embodiments, the methods and compositions of the present disclosure may be used to treat and/or prevent systemic inflammatory response syndrome (SIRS). SIRS is inflammation affecting the whole body. Where SIRS is caused by an infection, it is referred to as sepsis. SIRS may also be caused by non-infectious events such as trauma, injury, burns, ischemia, hemorrhage and/or other conditions. During sepsis and SIRS, complement activation leads to excessive generation of complement activation products which may cause multi organ failure (MOF) in subjects. The MUSCs and methods described herein may be used to control and/or balance complement activation for prevention and treatment of SIRS, sepsis and/or MOF. Additional methods of applying complement inhibitors to treat SIRS and sepsis may include those taught by Rittirsch et al. in Clin Dev Immunol, 2012, 962927, in U.S. publication No. U52013/0053302 or in U.S. Pat. No. 8,329,169, the contents of each of which are herein incorporated by reference in their entirety. Periodontitis

In some embodiments, the methods and compositions of the present disclosure may be used to treat or prevent development of periodontitis and/or associated conditions. Periodontitis is a widespread, chronic inflammation leading to the destruction of periodontal tissue which is the tissue supporting and surrounding the teeth. The condition also involves alveolar bone loss (bone that holds the teeth). Periodontitis may be caused by a lack of oral hygiene leading to accumulation of bacteria at the gum line, also known as dental plaque. Certain health conditions such as diabetes or malnutrition and/or habits such as smoking may increase the risk of periodontitis. Periodontitis may increase the risk of stroke, myocardial infarction, atherosclerosis, diabetes, osteoporosis, pre-term labor, as well as other health issues. Studies demonstrate a correlation between periodontitis and local complement activity. Periodontal bacteria may either inhibit or activate certain components of the complement cascade. Accordingly, the methods and compositions of the present disclosure may be used to prevent and/or treat periodontitis and associated diseases and conditions. Complement activation inhibitors and other treatment methods may include any of those taught by Hajishengallis in Biochem Pharmacol. 2010, 15; 80(12):1 and Lambris or in US publication No. U52013/0344082, the contents of each of which are herein incorporated by reference in their entirety.

Dental/Oral Trauma: In some embodiments, the methods and compositions of the present disclosure may be used to treat and/or promote healing of dental or oral trauma. Oral/dental traumas can include, but are not limited to wounds or injuries characterized by harm, damage or destruction caused by external events affecting the oral cavity, oral mucosa, tongue, teeth, gums, etc. Wounds can also be associated with cuts, blows, burns and/or other impacts to the oral cavity. Examples of such trauma include wear on the teeth, broken or fractured teeth, bitten tongue, burns of oral mucosa, cuts in gums and/or other injuries. Such traumas may be minor or severe. Often, dental or oral trauma, wounds and/or injuries are often acute, but if not healed properly they may lead to acute or chronic complications, local or systemic inflammatory responses, and/or infections and/or sepsis due to introduction of a pathogen (e.g., COVID-19). The methods of the present disclosure may be used to treat such trauma and/or to reduce or prevent related secondary complications of the trauma.

Anti-Phospholipid Syndrome (APS) and Catastrophic Anti-Phospholipid Syndrome (CAPS): In some embodiments, the methods and compositions of the present disclosure may be used to prevent and/or treat APS by complement activation control. APS is an autoimmune condition caused by anti-phospholipid antibodies that cause the blood to clot. APS may lead to recurrent venous or arterial thrombosis in organs, and complications in placental circulations causing pregnancy-related complications such as miscarriage, still birth, preeclampsia, premature birth and/or other complications. Catastrophic anti-phospholipid syndrome (CAPS) is an extreme and acute version of a similar condition leading to occlusion of veins in several organs simultaneously. Studies suggest that complement activation may contribute to APS-related complications including pregnancy-related complications, thrombotic (clotting) complications, and vascular complications. The methods and compositions described herein may be used to treat APS-related conditions by reducing or eliminating complement activation. In some cases, the MUSCs and methods described herein may be used to treat APS and/or APS-related complications. Other treatments for APS and APS-related conditions are taught by Salmon et al. Ann Rheum Dis 2002; 61(Suppl II):ii46-ii50 and Mackworth-Young in Clin Exp Immunol 2004, 136:393-401, the contents of which are herein incorporated by reference in their entirety.

Autoimmune Disease: The methods and compositions of the present disclosure may be used to treat subjects with autoimmune diseases and/or disorders. The immune system may be divided into innate and adaptive systems, referring to nonspecific immediate defense mechanisms and more complex antigen-specific systems, respectively. The complement system is part of the innate immune system, recognizing and eliminating pathogens. Additionally, complement proteins may modulate adaptive immunity, connecting innate and adaptive responses. Autoimmune diseases and disorders are immune abnormalities causing the system to target “self” tissues and substances. Autoimmune disease may involve certain tissues or organs of the body. MUSC compositions and the methods described herein may be used to modulate complement in the treatment and/or prevention of autoimmune diseases.

Pre-Eclampsia and HELLP-Syndrome: In some embodiments, methods and compositions of the present disclosure may be used to prevent and/or treat pre-eclampsia and/or HELLP (abbreviation standing for syndrome features of 1) hemolysis, 2) elevated liver enzymes and 3) low platelet count) syndrome by complement inhibitor therapy. Pre-eclampsia is a disorder of pregnancy with symptoms including elevated blood pressure, swelling, shortness of breath, kidney dysfunction, impaired liver function and/or low blood platelet count. Pre-eclampsia is typically diagnosed by a high urine protein level and high blood pressure. HELLP syndrome is a combination of hemolysis, elevated liver enzymes and low platelet conditions. Hemolysis is a disease involving rupturing of red blood cells leading to the release of hemoglobin from red blood cells. Elevated liver enzymes may indicate a pregnancy-induced liver condition. Low platelet levels lead to reduced clotting capability, causing danger of excessive bleeding. HELLP is associated with a pre-eclampsia and liver disorder. HELLP syndrome typically occurs during the later stages of pregnancy or after childbirth. It is typically diagnosed by blood tests indicating the presence of the three conditions it involves. Typically HELLP is treated by inducing delivery. Studies suggest that complement activation occurs during HELLP syndrome and pre-eclampsia and that certain complement components are present at increased levels during HELLP and pre-eclampsia. Complement inhibitors may be used as therapeutic agents to prevent and/or treat these conditions. Additional methods of preventing and/or treating HELLP and pre-eclampsia are taught by Heager et al. in Obstetrics & Gynecology, 1992, 79(1): 19-26 or in International publication No. WO201/078622, the contents of each of which are herein incorporated by reference in their entirety.

Atherosclerotic cardiovascular disease (ASCVD) is a complex disease involving multiple biological pathways, including the immune system and inflammatory response, and remains the primary cause of morbidity and mortality worldwide. Despite appropriate evidence-based treatments for patients with ASCVD, recurrence and mortality rates remain at approximately 2-4% per year. Risk factors for ASCVD can sometimes be attributed to genetic background and environmental factors, which together can lead to individual variations in response to therapy. Atherosclerotic disease is also influenced by the complex nature of the cardiovascular system itself where anatomy, function and biology all play important roles in health or disease. Atherosclerotic plaque consists of accumulated intracellular and extracellular lipids, smooth muscle cells, connective tissue, and glycosaminoglycans. The earliest detectable lesion of atherosclerosis is the fatty streak, consisting of lipid-laden foam cells, which are macrophages that have migrated as monocytes from the circulation into the subendothelial layer of the intima, which later evolves into the fibrous plaque, consisting of intimal smooth muscle cells surrounded by connective tissue and intracellular and extracellular lipids.

Coronary artery disease (CAD): is a narrowing or blockage of the arteries and vessels that provide oxygen and nutrients to the heart. It is caused by atherosclerosis, an accumulation of fatty materials on the inner linings of arteries. The resulting blockage restricts blood flow to the heart. When the blood flow is completely cut off, the result is a heart attack. CAD is the leading cause of death for both men and women in the United States. Atherosclerosis (also referred to as arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Inflammation occurs in combination with lipid accumulation in the vessel wall, and vascular inflammation is with the hallmark of atherosclerosis disease process.

Myocardial infarction is an ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in vessels previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Myocardial infarction (both ST and non-ST elevation) represents an unstable form of atherosclerotic cardiovascular disease. Acute coronary syndrome encompasses all forms of unstable coronary artery disease. Heart failure can occur as a result of myocardial dysfunction caused by myocardial infraction. Angina refers to chest pain or discomfort resulting from inadequate blood flow to the heart. Angina can be a symptom of atherosclerotic cardiovascular disease. Angina may be classified as stable, which follows a regular chronic pattern of symptoms, unlike the unstable forms of atherosclerotic vascular disease. The pathophysiological basis of stable atherosclerotic cardiovascular disease is also complicated but is biologically distinct from the unstable form. Generally stable angina is not myocardial necrosis.

Genetic Risk. As used herein, the term “an individual carrying at least one genetic risk factor” refers to humans in which one or more risk alleles are present in the genome. Such individuals have been shown to have an increased risk of: early onset myocardial infarction, abominal aortic aneurysm, stroke, peripheral artery disease, and myocardial infarction/coronary heart disease. This risk is independent of traditional risk factors, including diabetes, hypertension, cholesterol, and obesity. See, for example, Helgadottir et al. Science. 2007; 316(5830):1491-1493; Helgadottir et al. Nat Genet. 2008; 40(2):217-224; Palomaki et al. JAMA. 2010; 303(7):648-656; and Roberts et al. Curr Opin Cardiol. 2008; 23:629-633, each herein specifically incorporated by reference.

### Methods

In some embodiments, the therapeutic dosage can range from about 0.25 mL, 0.5 mL, 0.75 mL, 1.0 mL, 1.5 mL or 2 mL of a commercial solution of multipotent stem cells. The dosage may be adjusted for the concentration of the commercial product. An exemplary treatment regime entails administration daily, semi-weekly, weekly, once every two weeks, once a month, etc. In another example, treatment can be given as a continuous infusion. Therapeutic entities of the present disclosure are usually administered on multiple occasions. Intervals between single dosages can be weekly, monthly or yearly. It will be understood by one of skill in the art that such guidelines will be adjusted for the concentration of multipotent stem cells in the commercial product, in the use of co-therapeutic agents (e.g. anti-coagulant agents, anti-inflammatory agents, antibiotics, antiviral agents, statins, etc.).

For the treatment of disease, the appropriate dosage of the multipotent stem cells of the present disclosure vary depending upon many different factors, including the concentration of the composition, physiological state of the patient, whether the patient is human or an animal, other medications administered, the severity and course or stage of the disease, the patient's clinical history and response to the previous therapeutic compositions and/or delivery systems, whether the multipotent stem cells are administered for preventive/prophylactic purposes or to ameliorate symptoms of an extant disease state, adjunctive therapy, and the discretion of the attending physician. The multipotent stem cells are suitably administered to the patient at one time or over a series of treatments. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the stem cell composition is used in combination with another therapeutic agent, e.g., drugs useful in the treatment of atherosclerosis or diabetes, for example. Such combinations may include, without limitation, statins. Statins are inhibitors of HMG-CoA reductase enzyme. These agents are described in detail; for example, mevastatin and related compounds as disclosed in U.S. Pat. No. 3,983,140; lovastatin (mevinolin) and related compounds as disclosed in U.S. Pat. No. 4,231,938; pravastatin and related compounds as disclosed in U.S. Pat. No. 4,346,227; simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional agents and compounds are disclosed in U.S. Pat. Nos. 5,208,258, 5,130,306, 5,116,870, 5,049,696, RE 36,481, and RE 36,520. Statins include the salts and/or ester thereof. Other drugs that may be useful in combination include, for example, drugs to reduce hypertension, insulin, methotrexate, etc.

Optionally, the multipotent stem cell composition may contain other pharmaceutically acceptable components, such a buffers, surfactants, antioxidants, viscosity modifying agents, preservatives and the like. Each of these components is well-known in the art. See, for example, U.S. Pat. No. 5,985,310, the disclosure of which is herein incorporated by reference. Other components suitable for use in the formulations of the present invention can be found in Remington's Pharmaceutical Sciences, Mace Publishing Company, Philadelphia, Pa., 17th ed. (1985). In an embodiment, the aqueous cyclodextrin solution further comprise dextrose, e.g., about 5% dextrose.

Aqueous suspensions contain the active material in admixture with excipients suitable for the manufacture of aqueous suspensions. Such excipients are suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

The multipotent stem cell compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Therapeutic formulations comprising the multipotent stem cells with or without one or more co-therapeutic agents of the disclosure may be prepared for storage. The agent composition will be formulated, dosed, and administered in a fashion consistent with good medical practice. The “therapeutically effective amount” of the agent to be administered will be governed by such considerations, and is the minimum amount necessary to treat, ameliorate or prevent one or more symptoms of periodontitis, and/or one or more immune or inflammatory diseases or disorders. The agent can be suitably administered by multiple doses over a period of time, particularly with declining doses of the agent. The agent need not be, but is optionally formulated with one or more agents that potentiate activity, or that otherwise increase the therapeutic effect.

A co-therapeutic agent may be administered with or in the multipotent stem cell composition. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The multipotent stem cells of the present disclosure can be administered in the form of an injection into the oral vasculature and/or into a tooth pocket within the oral cavity. The pharmaceutical compositions are generally formulated as sterile, substantially isotonic and in full compliance with all Good Manufacturing Practice (GMP) regulations of the U.S. Food and Drug Administration.

Toxicity of the agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the multipotent stem cells described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

### Genetic Screening

In one aspect of the present disclosure, an individual is tested for the presence of a certain risk allele prior to treatment. Such methods comprise an analysis of genomic DNA in an individual for the allele that confers an increased susceptibility to one of the systemic diseases or conditions described herein. Individuals are screened by analyzing their genomic sequence at particular locations of the genome associated with a particular disease state, or at a representative SNP sequence for the presence of a predisposing allele, as compared to a normal sequence.

The presence of a predisposing risk allele is indicative that an individual is at increased risk of developing atherosclerosis and may benefit from treatment by the methods of the disclosure, although the methods can additionally find use in individuals without a genetic risk factor. The diagnosis of a disease predisposition allows the affected individual to seek early treatment of potential lesions, and to avoid activities that increase risk for cardiovascular disease

A number of methods are used for determining the presence of a predisposing variant in an individual. Genomic DNA is isolated from the individual or individuals that are to be tested. DNA can be isolated from any nucleated cellular source such as blood, hair shafts, saliva, mucous, biopsy, feces, etc.

### Assays

The umbilical stem cells can, for example, act as the basis for amelioration of such cardiovascular diseases as atherosclerosis, ischemia/reperfusion, hypertension, restenosis, and arterial inflammation. Such compounds may include, but are not limited to peptides, antibodies, or small organic or inorganic compounds.

Any technique known in the art may be used to introduce a target gene transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc.

With regard to intervention, any multipotent stem cell treatments that reverse any aspect of systemic disease symptoms should be considered as candidates for human disease therapeutic intervention. Dosages of test agents may be determined by deriving dose-response curves.

The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Ayass and OralDNA/MyPerioPath analyses: In addition to the forementioned parameters, additional indicators of disease may be detected using these tests. For example, cellular and extracellular debris produced by the process of apoptosis may be observed. Such uncleared debris has been found to be associated with autoimmune diseases. The methods described herein provides the means to clear or assist in the clearing debris and other sequelae of apoptosis. Gene expression profiling can also be used to identify significantly up-regulated and down-regulated genes as well as oral pathogen load pre- and post-injection to assess efficacy of treatment.

### EXPERIMENTAL

The following examples are put forth so as to provide those of ordinary skill in the art with a complete disclosure and description of how to make and use the present invention, and are not intended to limit the scope of what the inventor(s) regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. The present disclosure describes particular embodiments found or proposed by the present inventor(s) to comprise preferred modes for the practice of the disclosed subject matter. It will be appreciated by those of skill in the art that, in light of the present disclosure, numerous modifications and changes can be made in the particular embodiments exemplified without departing from the intended scope of the invention. Any recited method can be carried out in the order of events recited or in any other order which is logically possible.

All publications and patent applications cited in this specification are herein incorporated by reference as if each individual publication or patent application were specifically and individually indicated to be incorporated by reference.

### Example 1

Approximately 250 samples of patients' blood, saliva and/or other bodily fluids were collected in a dental clinic. Samples were assessed using Ayass and MyPerioPath testing and incorporating the assay results into a clinical calculator (See FIGS. 1-6) to measure biomarkers of disease and/or indicators such as antiphospholipid antibodies suggesting disease states or high-risk status for diseases such as APS, diabetes, etc. and to identify patients who were at low, moderate, or high risk of previously undetected disease, allowing the guided development of a multisystem health and wellness ITP.

For example, evidence has been obtained, using the methods and systems described herein, of stabilization of insulin/sugar levels observed as a post-injection decrease in hemoglobin A1C in a diabetic and edentulous patient not presenting current periodontal disease.

### Case Study 1: Ayass Testing and Results

Patient “CD” was a 52 year old female who was experiencing some maxillofacial pain and minor symptoms of periodontitis. Before and after treatment according to the methods and system disclosed herein, Ayass testing was conducted (see FIG. 2).

Data interpretation and findings, as well as recommended traditional treatments provided:
1. The presence of antibodies (DRVVT CONFIRM, SILICA CLOTTING TIME CONFIRM, ANTI-HISTONE), which coexist(s) with lupus erythematosus or incomplete lupus or undifferentiated connective tissue disease. The presence of undifferentiated connective tissue disease and incomplete lupus carries a high tendency (up to 50%) of developing into the full disease. Current measures may require initiation of therapy prophylactically like using immune remodeling therapy, Plaquenil to decrease the chance of full-blown systemic involvement like kidney failure, lung failure, and heart involving and coronary artery disease.
2. Positive antiphospholipids aPL antibody (DRVVT CONFIRM, SILICA CLOTTING TIME CONFIRM). This indicates the presence of autoimmune disease, undifferentiated connective tissue disease. These coexist with increased chance of clotting, VTE, deep venous thrombosis, pulmonary embolism, stroke, and heart attack, and may also coexist with the presence of malignancy or tumor overgrowth. This mandates a full cardiovascular workup, and may also mandate therapeutic measures like blood thinner, plus or minus immune remodeling therapy like Plaquenil.
3. Laboratory finding needs to be repeated within 6 weeks.
4. APS with positive aPL antibodies carries the risk of arterial and or venous thrombosis, valve disease, coronary artery disease, intra-cardiac thrombus formation, pulmonary hypertension and dilated cardiomyopathy. The most commonly affected valve is the mitral, followed by the aortic and tricuspid valve. This mandates a full cardiovascular workup.
5. The presence of aPL has significant systemic involvement in coexisting thrombosis and microcirculation that may affect the neuro, cardiac, kidney, and heart.
6. The presence of aPL correlates significantly with atherosclerosis disease and increased chance of myocardial dysfunction and heart attack.
7. The presence of aPL coexists with chronic fatigue and fibromyalgia.
8. The presence of aPL significantly correlates with neuropsychiatric disorder including seizures and ADHD.
9. The presence of aPL coexists with underlying autoimmune disease, connective tissue disease, lupus erythematosus (up to SO %) with monitoring in the following 10 years.
10. The presence of aPL indicates high risk of thrombosis (up to 50%) in the next 50 years.
11. The presence of aPL may mandate early therapeutic measures like immune remodeling therapy, such as Plaquenil, plus minus initiation of prophylactic anticoagulation with the use of Eliquis, Xarelto, and Coumadin.
12. The presence of aPL indicates coexistence of or put the patient at high risk of atherosclerosis disease in general and increases the intimal medial thickening in the carotid in association with plaque formation.
13. A significantly increased prevalence of Renal Artery Stenosis (RAS) (26%) was found in patients, with APS and hypertension.
14. Anti phospholipid syndrome (APS) associated with infections.
15. Antiphospholipid syndrome (APS) increases the frequency and severity of cardiac valvular disease.
16. Elevated aPL antibody titer is a Stroke Risk Factor.
17. The presence of antiphospholipids aPL mandates evaluation for neuropsychiatric findings, especially in patients taking medications for affected disorders such as depression or psychotic like schizophrenia, and may be accompanied by microthrombosis in the brain, mandating MRI for comprehensive evaluation of neurologic status.
18. The presence of antiphospholipids aPL mandates full evaluation of the thyroid panel, including thyroglobulin, thyroglobulin antibodies, and TPO antibodies, for evaluation of thyroiditis.
19. Patient has the presence of coagulopathy with activation of variant coagulation factors (FACTOR II ACTIVITY, FACTOR XII ACTIVITY), which coexist with increased patient's risk of thrombosis.
20. Patient has increased serum transferrin, which indicates inflammatory process.
21. Checking lipase and amylase, uric acid, electrolyte, special chemistry, hemoglobin Ale, and lipid levels helps to identify the environment that a patient has been exposed to due to alcoholic beverages, fatty foods, coffee, or food material with high amounts of uric acid. (CHOLESTEROL, TRIGLYCERIDES, LDL (CALCULATED), NON HDL CHOLESTEROL)
22. Elevated APOLIPOPROTEIN B/AI indicates increased risk of atherosclerosis disease and coronary disease and the presence of coexisting dyslipidemia.
23. Elevated APOLIPOPROTEIN B/AI RATIO in addition to the presence of aPL coexist with metabolic syndrome (MetS).
24. Patient has increased LACTATE serum level, which increases the chance of atherosclerosis and cardiac dysfunction.
25. Low vitamin D deficiency, which is highly prevalent in the general population, which indicates mal-preparedness in the immune system.

Based on findings for patient “CD” a full ITP according to the methods and systems described herein was designed and prescribed. Patient indicated intention to comply with ITP and to return for follow-up.

### Example 2

### Case Study 2

Young patient “CF” is a 16 y.o. male on diagnosed with autism spectrum disorder and having serious periodontal disease. Ayass and OralDNA MyPerioPath testing is performed to assess baseline parameters. Digital images obtained by intraoral camera are recorded and stored in a local database. Patient is assessed pre-injection using a 3D clinical calculator or nomogram similar to that illustrated in FIG. 1. An ITP is designed an prescribed, including an immediate, chairside injection of multipotent stem cells according to the methods disclosed herein. After application of a numbing agent, 1 mL of a composition consisting essentially of multipotent stem cells is IV-injected into a vein in the oral cavity using an 8 gauge needle. At one week post-treatment, the same parameters are re-measured. Some improvements in the symptoms of periodontitis are found to be improved, and the bacterial load is found to be reduced. A second ITP is designed to account for the rapidly observed progress, and a follow-up course of traditional dental cleanings, blood work and medical treatments prescribed.

### Example 3

### Case Study 3

Mature patient SM is a 53 y.o. female with significant oral plaque and some gum inflammation, multiple fillings and a crowns. She has no history of heart disease. A possible indication of autoimmune disease is noted. She is a former smoker, having ceased smoking more than 20 years ago. She reports having had a right nephrectomy at age 19, and some history of autoimmune disease including the presence of anti-CL antibodies, which was successfully treated with Plaquenil. She was also given subcutaneous Lovenox during a pregnancy to prevent adverse outcomes. She currently present with pre-hypertension, having a blood pressure measured in the dental clinic at 130/89. Ayass and OralDNA MyPerioPath testing is performed to assess baseline parameters and P.g. infection is detected and bacterial load quantified, and recorded as a previously undiagnosed risk of Alzheimer's disease. Digital images obtained by intraoral camera are recorded and stored in a local database. Patient is assessed pre-injection using a 2D nomogram similar to that illustrated in Table 1. An ITP is designed and prescribed, including a course of IV-injections of multipotent stem cells according to the methods disclosed herein and beginning with an immediate, chairside injection. After application of a numbing agent, 1 mL of a composition consisting essentially of multipotent stem cells is IV-injected into a vein in the oral cavity using an 8 gauge needle. Follow-up visits are scheduled for every six months to re-assess post-treatment parameters including those in the dental office as well as Ayass and OralDNA MyPerioPath testing, and self-assessment and reporting of cognitive function. While some improvements in the parameters measured by Ayass and OralDNA MyPerioPath testing may be found to be improved, and the bacterial load reduced, the attending dentist will continue to pay particular attention to any indications of symptoms indicating onset of Alzheimer's disease. Further genetic screening is recommended, and ongoing visits with specialists is prescribed in an updated ITP, and with a goal of reducing risk and possibly preventing onset of Alzheimer's disease, as well as enhanced multisystemic health and wellness and increased lifespan.

### Example 4

### Case Study 4

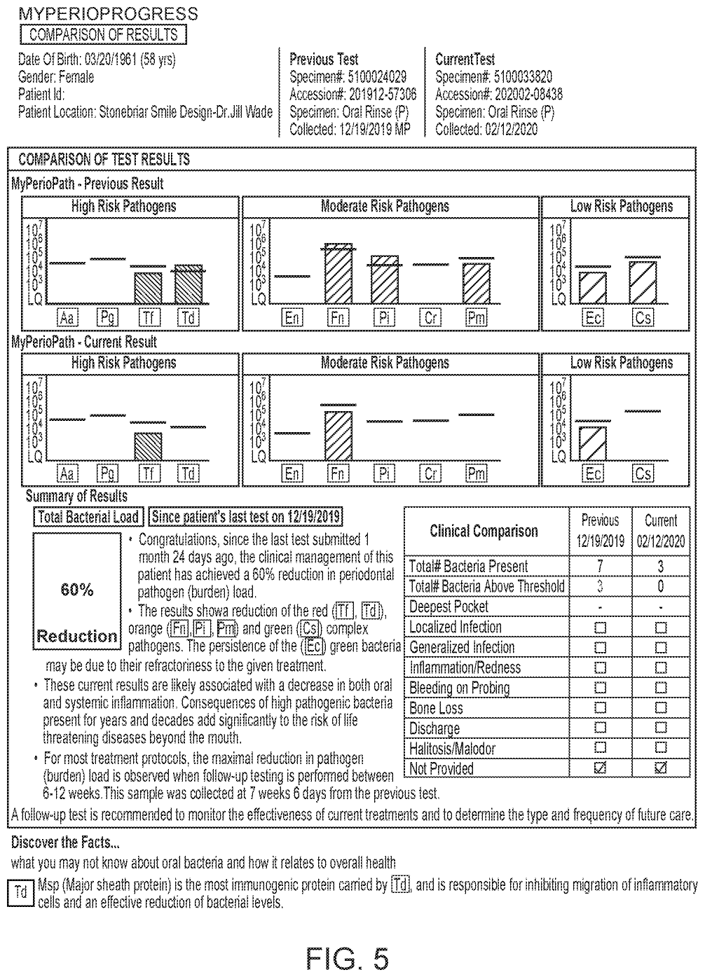

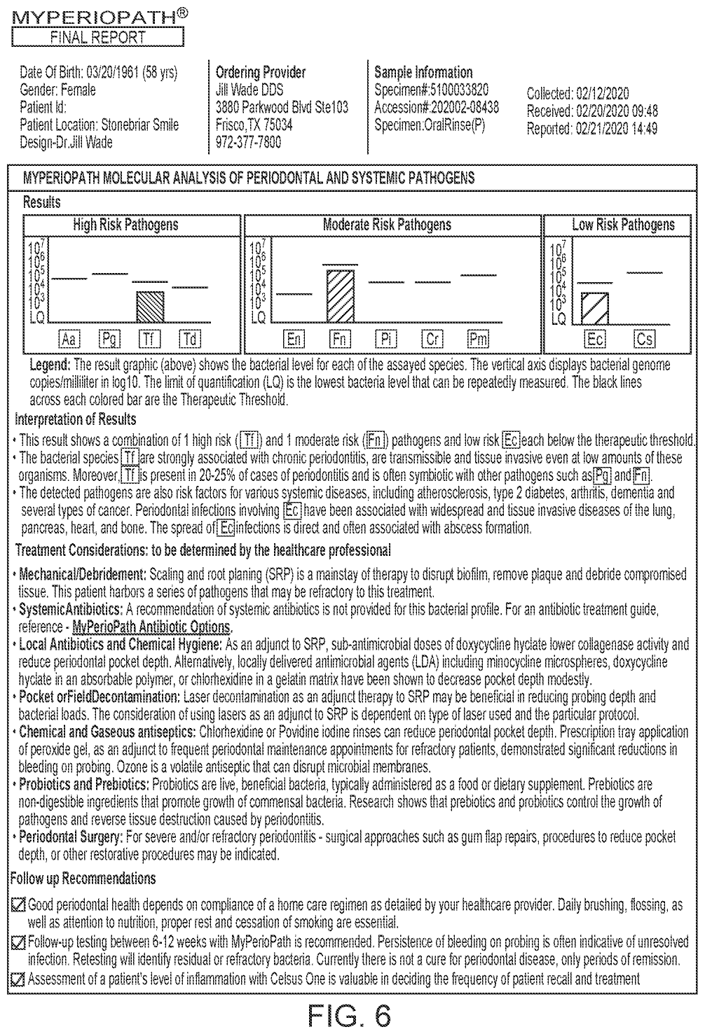

A 58 year old white female had a family history of Alzheimer's disease (her mother was currently living with symptoms of AD) presented clinically with multiple crowns and gum inflammation, including increased pocket depths and bleeding points, and positive results on Ayass and OralDNA pathogen tests (the presence of biomarkers of inflammatory disease and increased bacterial load). The patient was intraorally, intravascularly injected with 1 cc of multipotent stem cells but not given any other periodontal therapy. Upon retesting of saliva 6 weeks after the injection, this patient was observed to have markedly reduced inflammation of the gums, decreased pocket depths and bleeding points and a reduction in bacterial load as measured by OralDNA Labs tests. (FIGS. 5 and 6).

### Example 5

### Case Study 5

A 40 year old white female presented with a history of asthma, lupus, Antiphospholipid Syndrome diagnosed after eight miscarriages, clotting issues and acute swelling, lung inflammation (including a “rattle”) and respiratory issues requiring an inhaler (bronchitis 3-4 times a year). In the patient' own words, the inhaler gave her “no relief.” In the dental clinic, she was observed to have periodontal disease and tooth infections. A full Ayass Bioscience laboratory workup was performed and showed inflammatory biomarkers, and 1 cc of multipotent stem cells were intraorally, intravascularly injected. Within 12 hours of the injection, the respiratory “rattle” was gone, and the patient has not had to use an inhaler and has not had bronchitis to date. Furthermore, although a root canal was to be performed one-week after the injection, the endodontist found no infection (she was not taking any antibiotics). Since the injection, both the tooth infection and lung symptoms have completely resolved.

1-23. (canceled)

24. An oral intravenous (IV) injectable composition comprising multipotent stem cells, a second therapeutic agent, and a pharmaceutically-acceptable carrier.

25. A method of using an oral IV injectable composition comprising multipotent stem cells to improve multisystem health and wellness in a human patient, comprising:
a) assaying a body fluid sample of blood, saliva, gingival fluid, or a combination thereof, for levels of at least one of:
1. anticardiolipin or antiphospholipid antibodies, or both;
2. glucose, insulin, hemoglobin A1c, or a combination thereof;
3. periodontal pathogens or bacterial load, or both;
4. levels of cytokines;
5. levels of immune cells, epithelial cells or both;
6. expression levels of biomarkers;
b) obtaining the patient's baseline periodontal status measurements, comprising assessment of at least two parameters selected from:
i. patient's age;
ii. tooth pocket depth;
iii. number and position of gum bleeding points upon probing;
iv. level of gum tissue inflammation;
v. full mouth series (FMX) digital radiogram;
vi. intraoral camera photos; and
c) according to the results of the assay(s) of a) and the assessment of b), using a clinical calculator to determine the patient's weighted score;
d) using the weighted score to prescribe an individualized treatment plan (ITP), wherein the ITP includes specification of a therapeutically effective amount of the composition for injection into the patient's oral vasculature; and
e) intravenously injecting the therapeutically effective amount of the composition into the patient's oral vasculature;
wherein the IV injection of the composition results in, upon reassessment, a measurable improvement in at least one of the data points of a) or the selected parameters of b).

26. The method of claim 25, wherein the ITP further comprises administering a second therapeutic agent, concurrently or sequentially.

27. The method of claim 26, wherein the second therapeutic agent is an inhibitor of Porphyromonas gingivalis (P.g.).

28. The method of claim 25, wherein the ITP further comprises prescription of regular periodontal cleaning and debridement in the patient beginning within 90 days of the oral IV injection.

29. The method of claim 25, further comprising:
f) obtaining post-injection periodontal status measurements and inputting into the clinical calculator to determine the patient's post-injection weighted score, wherein the post-injection periodontal status measurements comprise reassessment of the parameters selected in the baseline periodontal status measurements;
g) making a first comparison of the pre-injection baseline periodontal status measurements to the post-injection periodontal status measurements; and
h) optionally, making a second comparison of both the baseline periodontal status and the post-injection measurements for each parameter to an ideal value; and
i) using the comparison(s) for prescription of a revised ITP.

30. The method of claim 25, further comprising:
j) repeating IV injection of the composition approximately every 90 days over a one-year period until the patient has a desired percent-improvement over the baseline periodontal status measurements.

31. The method of claim 25, further comprising:
k) monitoring the improvement in the patient, as demonstrated by one or more assayed levels selected from: an increased level of anti-inflammatory cytokines; a decreased level of pro-inflammatory cytokines; a reduction in levels of antiphospholipid antibodies and/or other symptoms of APS; a reduction of anticardiolipin signaling; a decrease of blood pressure; and positive changes in pre- and post-injection tested parameters.

32. A method of treating a condition in a human patient, wherein the condition is selected from the group consisting of:
antiphospholipid syndrome (APS), a disease of the digestive tract (including the oral cavity), an autoimmune disease, a chronic inflammatory disease, viral or bacterial infection, Alzheimer's disease (AD), migraine, joint pain, arthritis, fibromyalgia, and diabetes;
said method comprising IV-injecting into oral vasculature a composition comprising multipotent stem cells in an amount effective to treat the condition in the human patient;
wherein effective treatment is observed as a decrease in or cessation of symptoms experienced by the patient.

33. The method of claim 32, wherein the viral infective agent is COVID-19.

34. The method of claim 32, wherein the condition is acute inflammation of the digestive tract.

35. The method of claim 32, wherein the condition is periodontal disease which results in bone degeneration/erosion around natural teeth or implants.

36. The method of claim 25, wherein the composition stimulates dental bone regeneration, as measured by at least one of: 2-dimensional or 3-dimensional x-rays, and a decrease in pocket depth.

37. The method of claim 25, wherein the composition reduces the symptoms of bacterial infection.

38. The method of claim 37, wherein the bacterial infection is mediated by Porphyromonas gingivalis (P.g.).

39. The method of claim 38, further comprising co-administration of a small molecule inhibitor of P.g.

40. The method of claim 25, comprising co-administering autologous plasma rich platelets (PRPs).

41. The method of claim 25, wherein:
the IV injection treats an early symptom of AD or dementia; the early symptom being treated is selected from cognitive dysfunction, memory loss, impaired speech, or a combination thereof; and the treatment is observed as a 20% improvement in a post-injection measurement over the pre-injection baseline periodontal status measurement.

42. The method of claim 29, further comprising:
recording and storing the pre-injection baseline periodontal status measurements of selected parameters and ideal values on a computer readable format;
allowing approximately 90 days to six weeks to pass, post-injection, then re-measuring the selected post-injection parameters;
retrieving the stored pre-injection baseline periodontal status measurements and/or ideal values from the computer readable format;
comparing the post-injection measurements to the pre-injection baseline periodontal status measurements and the ideal values; and
using the comparison to prescribe a second ITP, whereby the patient who follows the prescribed second ITP achieves specific subgoals over the course of the second ITP and progresses toward the ideal values until an at least 50% improvement is observed when post-injection measurements are compared to the pre-injection baseline periodontal status measurements.
